(19) United States
(12) Patent Application Publication
(10) Pub. No.: US 2012/0100560 A1
(43) Pub. Date: Apr. 26, 2012

(54) DEVICE FOR CAPTURE, ENUMERATION, AND PROFILING OF CIRCULATING TUMOR CELLS

(75) Inventors: Peter C. Searson, Baltimore, MD (US); Kwan Hyi Lee, Baltimore, MD (US); Konstantinos Konstantopoulos, Elliott City, MD (US); ZiQiu Tong, Baltimore, MD (US)

(73) Assignee: The Johns Hopkins University, Baltimore, MD (US)

(21) Appl. No.: 13/190,290

(22) Filed: Jul. 25, 2011

Related U.S. Application Data
(60) Provisional application No. 61/367,188, filed on Jul. 23, 2010.

(51) Int. Cl. G01N 33/574; C12M 1/34; B82Y 5/00; B82Y 15/00

(52) U.S. Cl. 435/7.23; 435/287.2; 977/774; 977/904

(57) ABSTRACT

Applications in nanomedicine, such as diagnostics and targeted therapeutics, rely on the detection and targeting of membrane biomarkers. The present invention, in one embodiment, utilizes quantitative profiling, spatial mapping, and multiplexing of cancer biomarkers using functionalized quantum dots. This approach provides highly selective targeting molecular markers for pancreatic cancer with extremely low levels of non-specific binding and provides quantitative spatial information of biomarker distribution on a single cell, which is important since tumors cell populations are inherently heterogeneous. The quantitative measurements (number of molecules per square micron) is validated using flow cytometry and demonstrated using multiplexed quantitative profiling using color-coded quantum dots.

### CROSS-REFERENCE TO RELATED APPLICATIONS

This application claims priority to U.S. Provisional Patent Application Ser. No. 61/367,188, filed Jul. 23, 2010, the contents of which are hereby incorporated by reference in its entirety.

### STATEMENT REGARDING FEDERALLY SPONSORED RESEARCH OR DEVELOPMENT

This invention was made with government support under grant numbers US54CA143868 and US54CA151838 awarded by the National Institutes of Health. The government has certain rights in the invention.

### FIELD OF THE INVENTION

The invention relates to the field of diagnostic testing for cancer. The device and method are useful for detection, stage forecasting and clinical management of cancer. All references cited herein are hereby incorporated in their entirety.

### BACKGROUND OF THE INVENTION

Profiling Cancer Biomarkers: The detection of cancer biomarkers is important for diagnosis, disease stage forecasting, and clinical management. Since tumor populations are inherently heterogeneous, a key challenge is the quantitative profiling of membrane biomarkers, rather than secreted biomarkers, at the single cell level. The detection of cancer biomarkers is also important for imaging and therapeutics since membrane proteins are commonly selected as targets. Many methods for detection of membrane proteins yield ensemble averages and hence have limited application for analysis of heterogeneous populations or single cells. Fluorescence-based methods allow detection at the single cell level, however, photobleaching presents a major limitation in obtaining quantitative information. Quantum dots overcome the limitations associated with photobleaching, however, realizing quantitative profiling requires stable quantum yield, monodisperse quantum dot-antibody (QD-Ab) conjugates, and well-defined surface chemistry (Resch-Genger et al., Nature Methods 5(9):763-775, 2008).

Circulating Tumor Cells: Circulating tumor cells (CTCs) are tumor derived epithelial cells in the peripheral circulation of cancer patients (Allard et al., Clin Cancer Res, 10(20):6897-904, 2004; Cristofanilli et al., N Engl J Med, 351(8):781-91, 2004; Fehm et al., Clinical Cancer Research, 8(7):2073-2084, 2002; Steeg Nature Medicine, 12(8):895-904, 2006). Detection of CTCs has emerged as a promising method for diagnosis and clinical management of cancer patients (Mostert et al., Cancer Treatment Reviews, 35(5):463-474, 2009; Pantel et al., Nat Rev Clin Oncol, 6(4):190-1, 2009; Mocellin et al., Clinical Cancer Research, 12(15):4605-4613, 2006; Sleijfer et al., Eur J Cancer, 43(18):2645-50, 2007; Sleijfer et al., Eur J Cancer, 43(18):2645-50, 2007; Witzig et al., Clinical Cancer Research, 8(5):1085-1091, 2002; Braun et al., N Engl J Med, 351(8):824-6 2004). The clinical benefit of CTC detection has been demonstrated in metastatic breast (Cristofanilli et al., N Engl J Med, 351(8):781-91, 2004; Cristofanilli et al., J Clin Oncol, 23(7):1420-30, 2005; Weigelt et al., Br J Cancer, 88(7):1091-4, 2003, prostate (de Bono, et al., Clin Cancer Res 14(19):6302-9, 2008; Wood et al. J Clin Oncol, 15(12):3451-7, 1997) and colorectal (Molnar et al., Dis Markers, 24(3):141-50, 2008; Molnar et al., Clin Cancer Res, 7(12):4080-5, 2001; Cohen et al., J Clin Oncol, 26(19):3213-21, 2008) cancer. Isolation of CTCs has been reported for pancreatic (Kurihara et al., J Hepatobiliary Pancreat Surg, 15(2):189-95, 2008), gastric (Mimori et al., Clin Cancer Res, 14(9):2609-16, 2008), bladder (Gazzaniga et al., Clin Cancer Res, 7(3):577-83, 2001), and lung (Pachmann et al., Clin Chem Lab Med, 43(6):617-27, 2005) cancers. Recent results suggest that the number of CTCs before and after treatment of prostate cancer is predictive for overall survival, and more helpful than PSA (prostate specific antigen) detection (de Bono et al., Clin Cancer Res 14(19):6302-9, 2008). Detection and enumeration of CTCs is a potential tool to improve early detection, disease stage forecasting, and clinical management of pancreatic cancer patients.

The first reports of tumor cells in the peripheral circulation date back to the 19th century (Ashworth, Australian Medical Journal, 14:146-147, 1869). Detection of CTCs, however, has remained challenging due to their low concentration in cancer patients (Krivacic et al., Proc Natl Acad Sci USA, 101(29):10501-4, 2004; Ross et al., Blood, 82(9):2605-10, 1993; Racila et al., Proc Natl Acad Sci USA, 95(8):4589-4594, 1998). In 1 mL of blood there are typically 4×10⁹−6×10⁹ red blood cells, 4×10⁶−10×10⁶ leukocytes (white blood cells), and 1.5×10⁸−4×10⁸ platelets. The ability to detect as few as one CTC mL⁻¹ is generally considered to be necessary for diagnosis and therapeutic management (Mostert et al., Cancer Treatment Reviews, 35(5):463-474, 2009; Sleijfer et al., Eur J Cancer, 43(18):2645-50, 2007).

Methods for Detecting CTCs: Methods for detection of CTCs can be classified as cytometric (whole cell based) or nucleic acid based (Mostert et al., Cancer Treatment Reviews, 35(5):463-474, 2009; Sleijfer et al., Eur J Cancer, 43(18):2645-50, 2007). Due to the low concentration, most assays employ enrichment steps to increase the CTC concentration. Enrichment steps are usually based on immunoseparation or morphometric criteria. Immunoseparations generally involve positive selection and typically employ magnetic beads coated with antibodies to CTC antigens. Morphometric enrichment is based on physical characteristics such as cell size and cell density. Specificity is an issue for both methods due to heterogeneity in physical characteristics and marker expression.

EpCAM: Immunoseparations (e.g. CellSearch™) generally use epithelial cell adhesion molecule (EpCAM), since most tumor cells are derived from epithelial cells. EpCAM is a calcium signal transducer (CD326) involved in cell-cell adhesion and is highly expressed in many epithelial carcinomas (Allard et al., Clin Cancer Res, 10(20):6897-904, 2004). Some systems (e.g. AdnaTest™) utilize both EpCAM and mucin-1 to account for the fact that these biomarkers are not expressed by all circulating tumor cells (Tewes et al., Breast Cancer Res Treat, 115(3):581-90, 2009). Mucin-1 is a high molecular weight transmembrane glycoprotein overexpressed in many cancers (e.g. breast, lung, ovary, prostate, pancreatic, colorectal, bladder, and gastric). The tumor specific cell antigen epidermal growth factor receptor 2 (HER2) is also used for enrichment (Sleijfer et al., Eur J Cancer, 43(18):2645-50, 2007). Immunoseparations involving negative selection are performed by targeting leukocyte markers such as CD45 (Jacob et al., Expert Rev Proteomics, 4(6):741-56, 2007).

Cytometric techniques are often based on analysis of the size and shape (pleomorphism) of the cells and their nuclei, along with the nuclear to cytoplasm ratio using fluorescence and bright field microscopy. Nuclear staining with 4′,6-diamidino-2-phenylindole (DAPI) and staining with immunocytochemical markers such as antibodies to cytokines are used for analysis. Since neither red blood cells nor platelets have a nucleus, it is the ability to distinguish CTCs from leukocytes that is important in pleomorphic-based methods.

The only FDA approved assay for CTC detection is the CellSearch™ assay (Veridex, LLC). A 7.5 mL blood sample is centrifuged and then incubated with magnetic beads coated with epithelial cell adhesion molecule (EpCAM). This cell population is then stained with DAPI and fluorescent antibodies for CD45 and cytokeratin (CK). Cells that are positive for DAPI and CK but negative to CD45 are selected for morphological analysis. A CTC count of greater than or equal to 5 mL⁻¹ is considered positive.

Microfluidic approaches to sampling blood on the milliliter scale have been challenging (Coner et al., Annual Review of Biomedical Engineering, 7:77-103, 2005; El-Ali et al., Nature, 442(7101):403-411, 2006). A microfluidic approach, utilizing an array of posts coated with anti-epithelial cell adhesion-molecule (EpCAM), has been used to capture CTCs (Nagrath et al., Nature, 450(7173):1235-9, 2007). While preliminary results have been relatively successful, the device is complex, sample processing requires several hours, and identification of CTCs is solely based on EpCAM, a protein not expressed by all CTCs. That is why, the recovery and purity of CTCs is at best suboptimal. The presently disclosed invention improves on the limitations of the prior art by using a two-step system in a microfluidic device that increases the probability of capture of a CTC using optimized flow rates and flow path for aligned single cells, followed by secondary binding to reduce false positives to result in visualization of single CTCs out of a biological specimen.

A major advantage of cytometric methods over nucleic acid-based methods for CTC detection is that the target cells can be further characterized since they are not lysed in the procedure. A disadvantage of the cytometric methods is that the analysis is largely subjective. We combine CTC capture for cell enumeration with molecular profiling using QD-Ab conjugates. Both components of the assay are combined in a microfluidic platform.

Pancreatic Cancer: Pancreatic cancer is the fourth leading cause of cancer death in the US (about 30,000 per year), and has the highest mortality rate in the Western world (American Cancer Society Facts and Figures 2009, American Cancer Society, Atlanta, Ga. 2009; Jemal et al., CA Cancer J. Clin. 58(2):71-96, 2008). The survival rate amongst pancreatic cancer patients is extremely low, primarily due to the fact that a large fraction (about 80%) of tumors are metastatic at the time of diagnosis (Yeo et al., Curr. Probl. Cancer 26(4):176-275, 2002; Hezel et al., Genes Dev. 20(10):1218-1249, 2006; Espey et al., Cancer 110(10):2119-2152, 2007). The overall median survival time after diagnosis is 2-8 months, and only 1-4% of all patients with pancreatic adenocarcinoma survive 5 years after diagnosis (Singh et al., Cancer Res. 64(2):622-630, 2004). One of the major hallmarks of pancreatic cancer is its extensive local tumor invasion and early hematogenous and lymphatic dissemination to distant organs. Therefore, development of assays, that permit the efficient capture, enumeration and quantitative profiling of CTCs from peripheral blood of pancreatic cancer patients, is urgently needed for diagnosis and clinical management of the patients.

### BRIEF SUMMARY OF THE INVENTION

The invention relates to a microfluidic platform device employing a two stage procedure for capture, enumeration and profiling of circulating tumor cells. In the first stage, multiple receptors are immobilized in microfluidic channels for capture of circulating tumor cells from a biological sample. The second stage utilizes quantum dot-antibody conjugates to allow quantitative profiling of biomarkers on the captured tumor cells. Taken together, these results are used in the detection, stage forecasting and clinical management of cancer. In one embodiment, the invention relates to a microfluidic device for the capture, enumeration and profiling of circulating tumor cells. In another embodiment, the invention relates to a method of determining the presence of cancer, including pancreatic cancer, in a subject. In another embodiment, the invention relates to a method of diagnosing early stage cancer, including pancreatic cancer, in a subject. In another embodiment, the invention relates to a method of monitoring the progress of treatment of cancer, including pancreatic cancer, in a subject. In another embodiment, the invention relates to a kit for screening a subject sample for the presence of circulating tumor cells.

In one embodiment, the present invention relates to an integrated, high throughput capture, enumeration and profiling of circulating tumor cells (CTCs). The invention includes a microfluidic platform to combine capture, enumeration, and profiling of CTCs. The invention uses multiple antibodies, selected for their specificity for recognized biomarkers for early cancer detection, for cell capture. Using quantum dot-antibody conjugates for a second binding step, the invention provides information on false positives and allows quantitative profiling of cancer biomarkers. The invention contributes to the detection, stage forecasting, and clinical management of cancer.

The microfluidic platform of the prior art has the disadvantages that the sample processing requires several hours and the identification of CTCs is solely based on EpCAM, which is a protein not expressed by all CTCs. The present invention, in one embodiment, comprise (1) integrated capture, enumeration, and profiling of circulating tumor cells, (2) microfluidic-based platform for capture of circulating tumor cells, (3) capture based on multiple antibodies, (4) capture platform design based on knowledge of role of adhesion forces, and shear flow, (5) quantum dot-antibody conjugates to eliminate false positives, (6) quantum dot-antibody conjugates for biomarker profiling at the single cell level.

In one embodiment, the present invention relates to a method of determining the presence of pancreatic cancer in a subject. The invention includes collection of a biological sample from a subject suspected of having pancreatic cancer. The sample is then passed through a microfluidic device wherein CTCs are captured using one antibody and quantification using a quantum dot-antibody conjugate in a second binding step. The profiling of the tumor cells is accomplished by binding to particular antibodies.

In one embodiment, the present invention relates to a method of diagnosing pancreatic cancer in a subject. The invention includes collection of a biological sample from a subject suspected of having pancreatic cancer. The sample is passed through a microfluidic device wherein CTCs are detected via capture and then quantified with a second step using quantum dot-antibody conjugates. The binding of CTCs using markers of pancreatic cancer results in identification of the presence and diagnosis of pancreatic cancer in the subject.

In another embodiment, the invention relates to a method of monitoring the progress of treatment of pancreatic cancer in a subject. The invention includes collection of a biological sample from a subject with pancreatic cancer. The sample is passed through a microfluidic device wherein CTCs are detected via capture and then quantified with a second step using quantum dot-antibody conjugates. If the number of captured and quantified cells decrease over the course of treatment, the progress of the cancer has decreased. If the number of captured and quantified cells increase over the course of treatment, the progress of the cancer has increased.

In one embodiment, the present invention relates to a test kit for diagnosing the presence of pancreatic cancer in a subject. The test kit comprises a microfluidic platform device.

### BRIEF DESCRIPTION OF THE DRAWINGS

FIG. 1. (a) Using photolithography, selectin-coated stripes of fixed width, W, and varying lengths, L, separated by a distance, S=100 μm, in the direction of flow were generated. (b) Schematic of the microfluidic device assembled on top of a micropatterned glass surface.

FIG. 2. Comparison between experimental data (symbols) and predictions (solid lines) from our probabilistic multi-bond model. Data illustrate the fraction of PSGL-1-expressing HL-60 cells rolling on different lengths, x, of P-selectin-coated patches varying from 6 to 160 μm in the direction of flow. The P-selectin site density was 800 molecules/μm², whereas the wall shear stress varied from 0.5 to 2 dyn/cm².

FIG. 3. Quantitative profiling of biomarkers for pancreactic cancer. (a) Fluorescence images of pancreatic cancer cells (Panc-1, MIA Paca-2, and Capan-1) and normal panreatic cells (HPDE) incubated with 20 pmol QD-Ab conjugates (Ab=PSCA, CLDN4, and MSLN). (b) Average fluorescence intensity for Panc-1 cells incubated with different concentrations of QD-aMSLN. (c) Average fluorescence intensity for Panc-1 cells were incubated with QD-aCLDN4 conjugates or PE(phycoerythrin)-aCLDN4 conjugates versus illumination time. (d) Fluorescence images for different concentrations of QDs confined between two glass slides with fixed area. (e) Average fluorescence intensity (normalized for exposure time) versus QD concentration. (f) Average biomarker density for PSCA, claudin-4 and mesothelin in the three pancreatic cancer cell lines. (g) Distribution of mesothelin expression levels over a Panc-1 cell. (h) Fluorescence image of claudin-4 on capan-1. (i) Quantitative linear profiling of the claudin-4 density across a capan-1 cell.

FIG. 4. (Top) Schematic of the MDAI platform, which consists of a 6-channeled microfludic device (light blue) reversibly assembled on top of a glass slide (dark blue). Perfusion of six different biological functionalities through distinct channels enables their immobilization on the glass surface. The number of micro-channels and their dimensions can easily be manipulated. The distance between channels will be at least 100 μm. (Bottom) Schematic of the CIMM platform. The inlet and outlet of the device are connected by 10 straight microfluidic channels shown in brown. The micropatterned regions presenting the six distinct biological functionalities (i.e. antibodies specific for EpCAM, MUC1, MUC3, MUC4, MUC16 and CEA) are orthogonal to the direction of fluid flow and are shown in different colors.

FIG. 5. Simulations predicting the critical patch length required to mediate 99.5% of target (PSGL-1 expressing HL-60) cell adhesion to a P-selectin-coated substrate. Our analysis predicts that 240 μm patches are necessary for HL60 cell capture at mₗ=200 PSGL-1/μm² and mᵣ=750 P-selectin/μm² at a wall shear stress of 0.5 dyn/cm².

FIG. 6. (a) Schematic illustration of QD conjugates for biomarker targeting: (QD-L-PEG) CdSe/(Cd,Zn)S QDs with 80 mol % MHPC and 20 mol % DPE-Peg 2k. (QD-L-COOH) QDs with 80 mol % MHPC, 15 mol % DPE-PEG2k, and 5 mol % DPE-PEG2k-COOH. (QD-L-Ab) QD-L-COOH covalently conjugated with an average of three targeting antibodies, per QD. (b) Particle size distributions for QD conjugates. (c) Zeta potential for QD conjugates. A zeta potential of about −10 mV minimizes aggregation and non-specific binding. (d) Absorbance and emission spectra for QD-L-PEG (Em. 623 nm) in water. (e) Quantum yield for QD conjugates in water.

FIG. 7. Quantitative profiling of biomarkers for pancreatic cancer. Fluorescence images of pancreatic cancer cells (Panc-1, MIA Paca-2, and Capan-1) and normal pancreatic cells (HPDE) incubated with 20 pmol QD-Ab conjugates (Ab=aPSCA, aCLDN4, and aMSLN).

FIG. 8. Saturation of membrane biomarkers. Average fluorescence intensity for Panc-1 cells incubated with different concentrations of QD-aMSLN. The error bars represent the standard error for measurements over at least 30 cells. The slope at lower concentrations is 1.0 confirming negligible non-specific binding or competitive binding. The plateau at 10 mmol QDs indicates saturation of MSLN at the surface.

FIG. 9. Stability of fluorescence in QDs and fluorophores. Average fluorescence intensity for Panc-1 cells incubated with QD-aCLDN4 conjugates or PE (phycoerythrin)-aCLDN4 conjugates versus illumination time.

FIG. 10. Calibration of QD fluorescence. (a) Fluorescence images for different concentrations of QDs confined between two glass slides with fixed area. Top row: 36, 360, 1087 QDs μm⁻², bottom row: 1813, 2513, 2900 QDs μm⁻². (b) Average fluorescence intensity (normalized for 0.5 s exposure time) versus QD concentration.

FIG. 11. Absolute expression levels for biomarkers for pancreatic cancer. Average biomarker density per μm² for PSCA, claudin-4 and mesothelin in the three pancreatic cancer cell lines obtained from the average fluorescence intensity per cell and the calibration curve. Data were obtained from at least 300 Capan-1 cells, 100 MIAPaCa-2 cells, and 50 Panc-1 cells. Error bars represent the standard error.

FIG. 12. Spatial distribution of biomarkers. (a) Spatial distribution of mesothelin expression levels over a Panc-1 cell (inset). (b) Quantitative linear profiling of the claudin-4 density across a capan-1 cell (inset). The profiles were along radial lines separated by 22.5° and normalized to the cell diameter.

FIG. 13. Multiplexed imaging of cancer biomarkers on MIAPaCa-2 cells. Absorbance and emission spectra for (a) QD(Em.524)-L-aCLDN4, (b) QD(Em.623)-L-aMSLN, and (c) QD(Em.707)-L-aPSCA. (d) Phase contrast microscope image for MIAPaCa-2 cells after incubation with the three QD-Ab conjugates. Fluorescence images obtained with (e) FTIC (517/40, green), (f) TRITC (605/40, red), and (g) NIR (665 LP, infra red) filters. (h) Average biomarker density per cell for PSCA, claudin-4 and mesothelin in MIAPaCa-2 cells measured simultaneously. Standard error obtained from 150 cells.

### DETAILED DESCRIPTION OF THE INVENTION

The presence of cancer cells in the peripheral blood circulation can be used to screen for cancer. Antibodies appropriate for binding biomarkers associated with a particular cancer are useful to identify the presence and/or the progress of an organ based tumor, such as pancreatic cancer. In cases where cancer cells can be detected, when phenotypic signs of cancer are absent, it will be possible to better provide early diagnosis of cancer in a subject. Detection of biomarkers on circulating tumor cells (CTCs), which may be relatively low in number and thus require sensitivity in testing for detection, will provide a great benefit in the identification of disease such that a suitable course of medical treatment can be formulated. Such a tool can also be useful to monitor the progress of treatment of the cancer in the subject, as the number of CTCs in a biological sample would be expected to decrease during treatment. A “biological sample” or “biological specimen” includes, without limitation, cell-containing bodily fluids, fluids, peripheral blood, tissue samples, tissue homogenates and any other source of rare cells that are obtainable from a subject, preferably. Methods for the collection of blood and processing for analysis are well known in the art. For example, see U.S. Pat. No. 6,645,731.

In order to effectuate and facilitate the early detection, profiling and clinical management of cancer, more sensitive and accurate means of detection of circulating tumor cells is needed. To this end, the inventors have created a microfluidic platform device and methods for detecting the presence of and diagnosis of cancer in a subject, and developed a test kit to conduct the diagnostic testing.

The invention relates to a microfluidic platform device which combines capture, enumeration and profiling of circulating tumor cells. “Capture” involves the initial binding of CTCs expressing a biomarker of interest using an antibody for that marker; “enumeration” involves counting the circulating tumor cells captured in the microfluidic channel, and profiling involves quantitative identification of biomarkers expressed by the captured tumor cell using quantum dot-antibody conjugates.

The device uses multiple receptors for cell capture in different lanes on the surface of the MDAI. The receptors adhere to a substrate which has been applied to the surface of the MDAI, which substrate may be selected from any substrate known to adhere binding agents. The captured cells are then bound with quantum dot-antibody conjugates, which are introduced into the microchannel after the CTCs are captured, to allow quantitative profiling of cancer biomarkers resulting in the detection, stage forecasting and clinical management of cancer. In one embodiment, the invention relates to a microfluidic device for the capture, enumeration and profiling of circulating tumor cells. In another embodiment, the invention relates to a method of determining the presence of cancer, including pancreatic cancer, in a subject. In another embodiment, the invention relates to a method of diagnosing cancer, including pancreatic cancer, in a subject. In another embodiment, the invention relates to a method of monitoring the progress of treatment of cancer, including pancreatic cancer, in a subject. In another embodiment, the invention relates to a kit for screening a subject sample for the presence of circulating tumor cells.

Microfluidic Device: The fabrication of the microfluidic platform for CTC capture involves two steps: (1) fabrication of a Microfluidic Device for Antibody Immobilization (MDAI), allowing a pattern of patches of antibodies, which can be monoclonal antibodies, applied to a substrate for binding the CTC, which substrate has been applied on a glass slide, or other appropriate surface, and (2) fabrication of a Cell Isolation Multi-channeled Microfluidic (CIMM) platform located on the functionalized glass slide, thereby creating an enclosed microchannel between the two, while maintaining an inlet and outlet. The channels on the CIMM are aligned perpendicular to the patches, such that the patch width in the MDAI is the patch length in the CIMM.

In one embodiment, the invention relates to a microfluidic device for capture of Circulating Tumor Cells (CTCs). The MDAI portion of the device may be crafted of glass, plastic or other suitable material conducive for patterning and adherence of biomarkers and visualization of bound cells under a microscope. Adherence of the binding partner to the surface of the MDAI may be accomplished by any means known in the art. Stripes of fixed width, about 1-5 μm in size, about 5-10 μm in size, about 10-15 μm in size, about 15-20 μm in size and about 20-50 μm in size, are patterned on a surface, for example a glass slide, using a modified photolithography technique. The stripes are spaced 100 μm apart from each other. The stripes can be spaced 50 μm apart, 100 μm apart, 150 μm apart or 200 μm apart. The spacing will be dependent on the number of stripes present on the surface as well as the physical dimensions of the device.

The length of the strips depends on the dynamic shear stress of the cells passing through the formed microchannel configuration. The inventors have found that the critical patch length for cell capture is about 40 μm at 2 dyn/cm², about 10 μm at 1 dyn/cm² and about 6 μm at 0.5 dyn/cm². Therefore, the length of the strip can be about 6-10 μm, about 10-20 μm, about 20-30 μm, about 30-40 μm, about 40-50 μm, about 50-60 μm, about 60-70 μm, about 70-80 μm, about 80-90 μm or about 90-100 μm. Any pump capable of maintaining a constant flow rate and maintaining the appropriate shear flow, is appropriate for the device. A syringe pump, for example, may be used with the device. Selection of the appropriate pump is well within the skill of one of ordinary skill in the art.

The modified photolithography technique involves using a positive photoresist to coat a pre-cleaned surface, for example a pre-cleaned glass slide, followed by subsequent exposure to ultraviolet light through a chrome mask patterned with an appropriate design corresponding to the desired number and dimensions of the stripes. The irradiated regions of photoresist are then dissolved upon incubation with MF-CD-26 Developer. The photoresist-patterned surface or glass slide is then immersed in 0.1% (v/v) solution of octadecyltrichlorosilane in hexane, thus rendering the surface hydrophobic.

The slide is then treated with a binding partner, for example, FITC-labeled goat anti-human IgG-Fc specific antibody, prior to addition of P- or L-selectin Ig chimeras, thus immobilizing the selectins to the surface or glass slide. Other methods of adhering an antibody to a surface, such as a glass slide, are known to those of ordinary skill in the art. Other binding partners, well known to those of ordinary skill in the art, may be used. Other methods of adhering the antibodies to the surface are also well known in the art. The micro-patterned protein patches appear after rinsing the slide with remover solution. The P- and L-selectin site density can be measured using the dissociation-enhanced lanthanide fluorescent immunoassay.

The CIMM portion of the device, which can be fabricated from polydimethylsiloxane (PDMS) or other suitable material, which materials are well within the knowledge of one of ordinary skill in the art, contains a plurality of microchannels, which depth is limited to enable single cell flow through the channel. The microchannel is about 8-10 μm, about 10-12 about 12-14 μm, about 14-16 μm, about 16-18 μm, about 18-20 μm, about 20-22 μm, about 22-24 μm, about 24-26 μm, about 26-28 μm or about 28-30 μm in depth. Limiting the depth of the flow channel ensures formation of a single file of cells over the micropatterned substrate and thus an accurate determination of cell flux on the surface.

The enclosed microchannels are formed by placing the CIMM over the MDAI and contain an inlet and outlet for the biological specimen to flow across the patterned surface on the MDAI to be captured by the appropriate binding partner. The binding partner for each channel can be a marker for a CTC. In one embodiment, the channels on the MDAI are coated individually with antibodies for EpCAM, MUC1, MUC3, MUC4, MUC16 and CEA. The antibodies utilized in the invention can be monoclonal antibodies. To illustrate the concept, channel 1 contains an antibody for EpCAM, channel 2 contains an antibody for MUC1, channel 3 contains an antibody for MUC3, channel 4 contains an antibody for MUC4, channel 5 contains an antibody for MUC16 and channel 6 contains antibody for CEA. Other binding partners, recognized by those of ordinary skill in the art, are suitable for use in the microfluidic device.

Staging of Pancreatic Cancer: The histologic progression from non-invasive precursor lesions (called Pancreatic Intraepithelial Neoplasia or PanINs) to invasive and metastatic pancreatic cancer is associated with the sequential accumulation of molecular markers (Maitra et al., Adv Anat Pathol 12(2):81-91, 2005; Maitra et al. Mod Pathol 16(9):902-912, 2003; Maitra et al. Annu Rev Pathol 3:157-188, 008; Prasad et al., Cancer Res 65(5):1619-26, 2005). For example, cell surface proteins such as prostate stem cell antigen (PSCA) (Maitra et al. Mod Pathol 16(9):902-912, 2003; Argani et al., Cancer Res 61(11):4320-4324, 2001) are aberrantly overexpressed even at the stage of non-invasive precursor lesions. In contrast, the protein mesothelin is aberrantly overexpressed only on the surface of infiltrating cancer cells, but not on the surface of normal pancreatic ducts or non-invasive PanINs (Maitra et al., Mod Pathol 15(1):137A, 2002; Maitra et al. Mod Pathol 16(9):902-912, 2003; Argani et al., Cancer Res 61(11):4320-4324, 2001; Argani et al., Clinical Cancer Res 7(12):3862-3868, 2001).

Three biomarkers are useful for identifying the stage of pancreatic cancer for quantitative imaging: prostate stem cell antigen (PSCA), claudin-4 (CLDN4), and mesothelin (MSLN). PSCA and MSLN are gylcosylphosphatidyl inositol (GPI)-anchored proteins whereas CLDN4 is one of a large family of tight junction proteins. PSCA is overexpressed in adenocarcinomas and present in the majority of PanIN lesions beginning with early PanIN-1 (Maitra et al., Modern Pathology 15(1):137A, 2002; Wente et al., Pancreas, 31(2):119-125, 2005). Claudin-4 overexpression is observed in intermediate PanIN-2 lesions (Michl, et al., Gastroenterology 121(3):678-684, 2001; Nichols et al., Am J Clinical Pathology 121(2):226-230, 2004; Morin, Cancer Res 65(21):9603-9606, 2005)). Mesothelin overexpression is a late event in the progression model of pancreatic cancer, almost always associated with invasion (Maitra et al., Modern Pathology 15(1):137A, 2002; Li et al., Molecular Cancer Therapeutics 7(2):286-296, 2008; Argani et al., Clinical Cancer Research 7(12):3862-3868, 2001). Therefore, if a CTC expresses only PSCA, it is an early stage pancreatic cancer, if a CTC expresses CLDN4, it is an intermediate stage pancreatic cancer, if a CTC expresses MSLN, it is a late stage pancreatic cancer. All three of these biomarkers are therapeutic targets for pancreatic cancer.

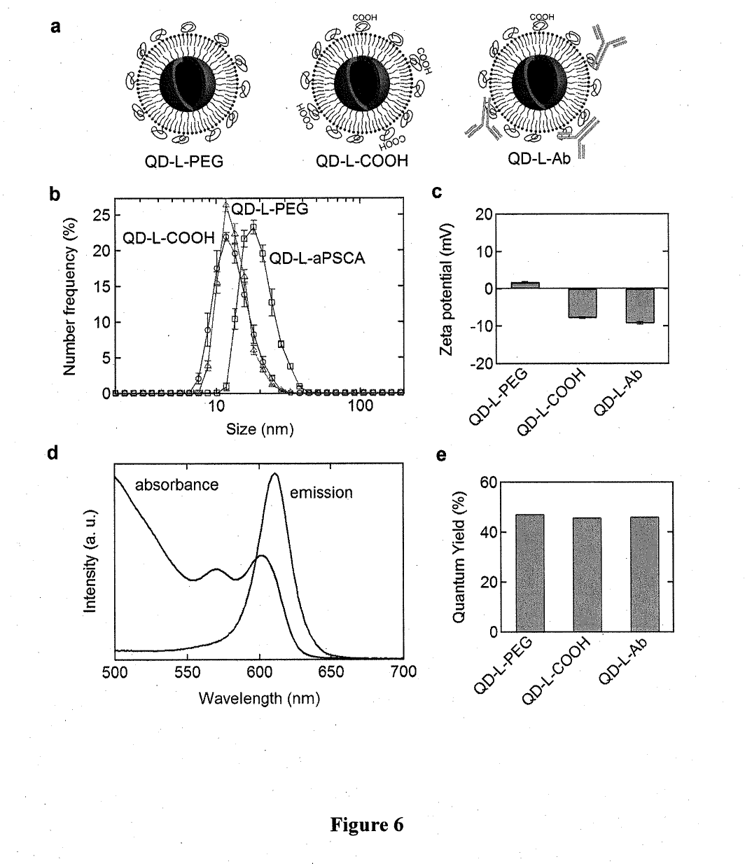

QDs exhibit size-dependent absorption and emission properties (Brus, J Chemical Physics 80(9):4403-4409, 1984), high fluorescence quantum yields, and with careful functionalization have been widely used for imaging and sensing (Michalet et al., Science 307(5709):538-544, 2005; Medintz et al., Nature Materials 4(6):435-446, 2005; Gao et al., Nature Biotechnology 22(8):969-976, 2004; Sapsford et al., Sensors 6(8):925-953, 2006; Choi et al., Nat Biotechnol 25(10):1165-1170, 2007; Gao et al., Bioconjug Chem 21(4):604-609, 2010; Fu et al., Current Opinion in Neurobiology 15(5):568-575, 2005; Ballou et al., Bioconjugate Chemistry 15(1):79-86, 2004; Smith, et al., Nano Letters 8(9):2599-2606, 2008). Quantitative QD-Ab targeting requires that each target molecule (e.g. membrane protein) is conjugated with one QD and that non-specific binding is minimized. Although various functionalization schemes have been reported in the literature (Medintz et al., Nature Materials 4(6):435-446, 2005; Gao et al., Nature Biotechnology 22(8):969-976, 2004; Dubertret et al., Science 298(5599):1759-1762, 2002; Liu et al., Acs Nano 4(5):2755-2765, 2010; Howarth et al., Nature Methods 5(5):397-399, 2008; Mulder et al. Accounts of Chemical Research 42(7):904-914, 2009; Louie et al., Chemical Reviews 110(5):3146-3195, 2010), here the inventors have developed a method based on encapsulation with a lipid layer (Dubertret et al., Science 298(5599):1759-1762, 2002; Cormode et al., Nano Letters 8(11):3715-3723, 2008; Carion et al., Nature Protocols 2(10):2383-2390, 2007; Koole et al., Bioconjugate Chemistry 19(12):2471-2479, 2008) optimized for quantitative targeting (FIG. 6a).

Synthesis of quantum dots (QDs): CdSe/(Cd,Zn)S core/shell QDs with an emission wavelength of about 610 nm are useful in the invention (Park et al., J Physical Chem 112(46):17894-17854, 2008; Galloway et al., Science of Advanced Materials 1(1):1-8, 2009). For multiplexing experiments, CdSe/(Cd,Zn)S core/shell QDs with an emission wavelength of 524 nm and CuInSe/ZnS core/shell QDs with an emission wavelength of 707 nm are useful.

Water soluble QDs are obtained by forming a lipid monolayer composed of MHPC/DPPE-PEG2k (80:20 mol %) or MHPC/DPPE-PEG2k/DPPE-PEG2k-COOH (80:15:5 mole %). Typically 0.25 nmol of QDs, 4 μmol of MHPC, 0.75 μmol of DPPE-PEG2k, and 0.25 μmol of DPPE-PEG2k-COOH are dissolved in 0.3 mL of chloroform. This solution is added to 2 ml of deionized water and heated and maintained at 110° C. for 1 h under vigorous stirring to evaporate chloroform. The resulting solution is sonicated for 1 h, centrifuged, and the supernatant then passed through a syringe filter with a 200 nm PTFE membrane (VWR) to remove any aggregates or unsuspended QDs. Quantum yield measurements are performed on suspensions with about 100 pmol QDs in 4 mL DI water using a Hamamatsu C9920-02 fluorometer.

In one embodiment, the present invention relates to an integrated, high throughput capture, enumeration and profiling of circulating tumor cells (CTCs). The invention relates to a microfluidic platform to combine capture, enumeration, and profiling of CTCs. The invention uses multiple antibodies, selected for their specificity for recognized biomarkers for early cancer detection, for cell capture and characterization using quantum dot-antibody conjugates. Initial capture of a CTC on the MDAI-bound antibody, followed by secondary binding of a quantum dot-antibody conjugate, reduces the incidence of false positives and allows quantitative profiling of cancer biomarkers. The invention contributes to the detection, stage forecasting, and clinical management of cancer.

The microfluidic platform of the prior art has the disadvantages that the sample processing requires several hours and the identification of CTCs is solely based on EpCAM, which is a protein not expressed by all CTCs. The present invention, in one embodiment, comprise (1) integrated capture, enumeration, and profiling of circulating tumor cells, (2) microfluidic-based platform for capture of circulating tumor cells, (3) capture based on multiple antibodies, (4) capture platform design based on knowledge of role of adhesion forces, and shear flow, (5) quantum dot-antibody conjugates to eliminate false positives, (6) quantum dot-antibody conjugates for biomarker profiling at the single cell level.

In one embodiment, the present invention relates to a method of determining the presence of pancreatic cancer in a subject. The invention includes collection of a biological sample from a subject suspected of having pancreatic cancer. The sample is then passed through a microfluidic device, comprising a MDAI and CIMM, wherein the MDAI is patterned and coated with antibodies for capture of CTCs. In one embodiment, the antibodies are individually applied in a channel. The antibodies can be EpCAM, MUC1, MUC3, MUC4, MUC16 and CEA. After passing the biological sample through the microfluidic device, CTCs expressing the marker for EpCAM, MUC1, MUC3, MUC4, MUC16 or CEA are captured in that channel coated with its binding partner. Next, quantum dot-antibodies are passed through the inlet to then bind those cells expressing those specific markers. The CTCs are thus detected via capture and quantified using quantum dot-antibody conjugates. The profiling of the tumor cells is accomplished by binding to particular antibodies associated with a specific cancer type.

In one embodiment, the present invention relates to a method of diagnosing pancreatic cancer in a subject. The invention includes collection of a biological sample from a subject suspected of having pancreatic cancer. The sample is passed through a microfluidic device wherein CTCs are detected via capture and quantified using quantum dot-antibody conjugates. The binding of CTCs using markers of pancreatic cancer results in identification of the presence and diagnosis of pancreatic cancer in the subject.

In one embodiment, the present invention relates to a test kit for diagnosing the presence of pcancer in a subject. The test kit comprises a microfluidic device comprising a MDAI and CIMM which contains an inlet and outlet for a biological sample. The kit can further comprise quantum dot-antibody conjugates for passage into the microchannel to bind captured CTCs from a subject.

The device of the invention is applicable for capture of any CTC, based on the binding agent being employed, and the second step binding using quantum dot-antibody conjugates is applicable to the use of any quantum dot-antibody conjugate. For purposes of illustration, but in no way limiting the scope of protection being sought, the invention in various embodiments is presented in the examples below.

### EXAMPLES

### Example 1

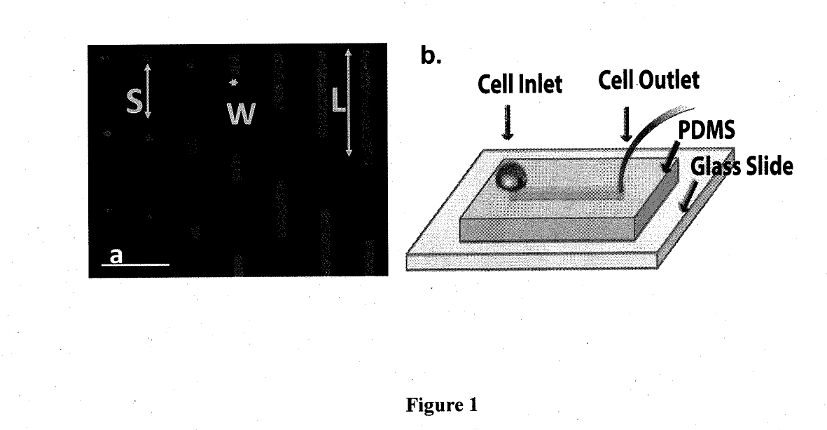

Development of a microfluidic device for selective capture of free-flowing cells on micro-patterned surfaces. Stripes of fixed width (10 μm) but varying lengths (6, 10, 20, 40, 80, 120 and 160 μm) separated by 100 μm in the direction of flow were patterned on a glass slide using a modified photolithography technique (FIG. 1a) (Ghosh et al. Langmuir 24(15):8134-42, 2008). In brief, a positive photoresist was used to coat a pre-cleaned glass slide surface, which was subsequently exposed to UV light through a chrome mask with the appropriate design. The irradiated regions of photoresist were then dissolved upon incubation with MF-CD-26 Developer. The photoresist-patterned slide was then immersed in 0.1% (v/v) solution of octadecyltrichlorosilane in hexane to render the slide surface hydrophobic. FITC labeled goat anti-human IgG-Fc specific antibody was then added to the slide prior to the immobilization of P- or L-selectin-Ig chimeras at prescribed concentrations (Ghosh et al. Langmuir 24(15):8134-42, 2008) to ensure the proper orientation of immobilized selectins. The micro-patterned protein patches appeared (FIG. 1a) after rinsing the slide with remover solution. The P- and L-selectin site density was measured using the dissociation-enhanced lanthanide fluorescent immunoassay, as previously described (Ham et al., Biotechnol Bioeng 96(3):596-607, 2007).

A single-channel microfluidic device (L×W×H=2 cm×800 μm×25 μm), fabricated from PDMS, was assembled on top of a selectin-micropatterned glass slide (FIG. 1b), and placed onto the stage of an inverted microscope. The 25 μm depth/height of the flow channel ensured the formation of a single file of cells over the micropatterned substrate, and thus the accurate determination of cell flux on the surface. Fluorescent light was used to visualize the protein-micro-patterned regions (FIG. 1a). 50 μL of P-selectin glycoprotein ligand-1 (PSGL-1)-expressing HL-60 promyelocytic leukemia cells were dispensed to the inlet of the microfluidic device, and perfused through the channel for 3 minutes at a prescribed flow rate via a syringe pump, which was connected to the outlet of the device. The extent of cell binding to individual patches and average rolling velocities as a function of wall shear stress were quantified, as previously reported.

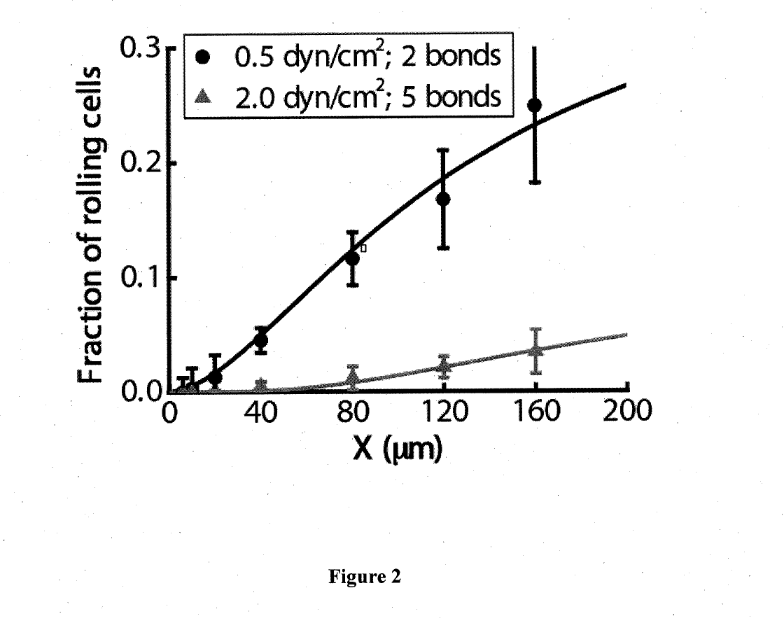

Our flow-based adhesion assays reveal: (1) that the extent of cell tethering decreases on increasing the shear stress from 0.5 to 2 dyn/cm² (FIG. 2), (2) the fraction of captured cells increases with increasing the patch length (FIG. 2), and (3) a critical patch length is required for cell capture, which is dependent on the site density and the level of applied shear stress. For instance, the critical patch length for cell capture is 40 μm at 2 dyn/cm², 10 μm at 1 dyn/cm², and 6 μm or less at 0.5 dyn/cm². Furthermore, reducing the P-selectin site density on the micro-patterned surface increases the critical patch length that is required for cell capture.

Cell adhesion depends on the balance between the dispersive hydrodynamic forces due to blood flow and the adhesive forces generated by the interactions between membrane-bound receptors and ligands anchored to apposing cell surfaces. The motion of both the receptor(s) and ligand(s) is thus restricted to 2-D (Chesla et al, Biophys J 75(3):1553-1572, 1998; Piper et al., Biophys J 74(1):492-513, 1998). As such, the 3-D kinetic constants determined by Surface Plasmon Resonance and radioimmunoassays are not relevant to describe the 2-D kinetics of receptor-mediated cell adhesion (Chesla et al, Biophys J 75(3):1553-1572, 1998). Experimental techniques based on fluorescence imaging of adhesion molecules (Dustin et al. J Biol Chem 272(49):30889-98, 1997) or micropipette aspiration techniques (Chesla et al, Biophys J 75(3):1553-1572, 1998) have been developed to address this issue. However, these methods fail to recapitulate the cell tethering behavior observed under physiologically relevant flow conditions. While flow chamber assays mimic the shear environment of the circulation, all of the previous studies have focused on the determination of the receptor-ligand bond dissociation rate constant (k_{off}) (Alon et al., Nature 374(6522):539-42, 1995; Smith et al., Biophysical J 77(6):3371-3383, 2004; Yago et al., J Cell Biol 166(6):913-923, 2004). To our knowledge, we have developed the first mathematical model to determine the 2-D binding kinetic constants and effective number of bonds from cell adhesion assays, based on the kinetics of receptor-ligand interactions (R+L [symbol] RL) and probabilistic modeling. Since cell adhesion can be mediated by any number of bonds ranging from 0 to A_{c}mₘᵢₙ, where A_{c} is the contact area between an interacting cell and the substrate and mₘᵢₙ=min(mᵣ, mₗ), where mᵣ and mₗ represent the site density of receptors and ligands, respectively, we calculate the probability of having n-bonds [P(0), P(1), . . . , P(n), . . . P(A_{c}mₘᵢₙ)] as a function of the cell capture distance on the micropatch. Assuming that the number of bonds is much smaller than the total number of available receptors and ligands, the solution to P(n) is of the form of the Poisson distribution as previously reported in the literature (Chesla et al, Biophys J75(3):1553-1572, 1998):

$${P}_{n} ( t ) = \frac{{〈 n 〉}^{n}}{n !} exp ( - 〈 n 〉 )$$

where <n>=A_{c}mᵣmₗkₒₙ/k_{off[}1−exp(−k_{off}t)] is the average number of bonds as a function of time for an interacting cell on the selectin-coated micropatch, and the time variable, t, will be substituted by t=x/U_{cell} to yield an expression in terms of rolling distance x where U_{cell} is the velocity of the cell over the protein-coated patch. The input parameters in our model are: (1) the fraction of interacting cells (N_{b}/N_{T}), which is measured experimentally at different patch lengths, (2) the force (F_{b}) exerted on the bond of the cell, which is estimated using the Goldman model (Alon et al., Nature 374(6522):539-42, 1995; Goldman et al. Chem Eng Sci 22(4):653, 1967), (3) the length of the lever arm (the distance between the tether point and the projection of the cell center on the substrate), which is measured experimentally from flow reversal assays, as previously described (Yago et al., J Cell Biol 166(6):913-923, 2004; Alon et al. J Cell Biol 138(5):1169-80, 1997; Yago et al., J Cell Biol 158(4):787-99, 2002), (4) the receptor (mᵣ) and ligand (mₗ) site densities, which are measured experimentally, and (5) k^{o}_{off} and x_{β}, which are measured by atomic force microscopy (AFM). The optimization parameters A_{c}mᵣkₒₙ and number of bonds, n, are obtained by minimizing the sum of squares of the residual between the theoretical prediction and experiment data. We have applied our multi-bond model to investigate the rolling of PSGL-1-expressing HL-60 cells to immobilized P-selectin in shear flow. As shown in FIG. 2, there is an excellent agreement between the theoretical predictions and experimental data. Our analysis also reveals that 2 and 5 PSGL-1-P-selectin bonds are necessary to mediate HL-60 cell rolling on immobilized P-selectin at 0.5 and 2 dyn/cm², respectively. The affinity constant)(Kₐ^{o}) of the P-selectin-PSGL-1 bond determined from our experimental/mathematical approach is 2.3×10⁻³ μm², which in excellent agreement with the value calculated from previous single-bond experimental data (2.9×10⁻³ μm²) (Lawrence et al., J Cell Biol 136(3):717-27, 1997).

### Example 2

Quantitative profiling of molecular biomarkers for pancreatic cancer. In preliminary studies we have demonstrated proof-of-principle of quantitative profiling of mesothelin, claudin-4, and PSCA in three pancreatic cancer cells lines. The histologic progression from non-invasive precursor lesions (called Pancreatic Intraepithelial Neoplasia or PanINs) to invasive and metastatic pancreatic cancer is associated with the sequential accumulation of molecular markers (Maitra et al., Adv Anat Pathol 12(2):81-91, 2005; Maitra et al. Mod Pathol 16(9):902-912, 2003; Maitra et al. Annu Rev Pathol 3:157-188, 008; Prasad et al., Cancer Res 65(5):1619-26, 2005). For example, cell surface proteins such as prostate stem cell antigen (PSCA) (Maitra et al. Mod Pathol 16(9):902-912, 2003; Argani et al., Cancer Res 61(11):4320-4324, 2001) are aberrantly overexpressed even at the stage of non-invasive precursor lesions. In contrast, the protein mesothelin is aberrantly overexpressed only on the surface of infiltrating cancer cells, but not on the surface of normal pancreatic ducts or non-invasive PanINs (Maitra et al., Mod Pathol 15(1):137A, 2002; Maitra et al. Mod Pathol 16(9):902-912, 2003; Argani et al., Cancer Res 61(11):4320-4324, 2001; Argani et al., Clinical Cancer Res 7(12):3862-3868, 2001). These molecular markers are ideal targets for quantitative profiling.

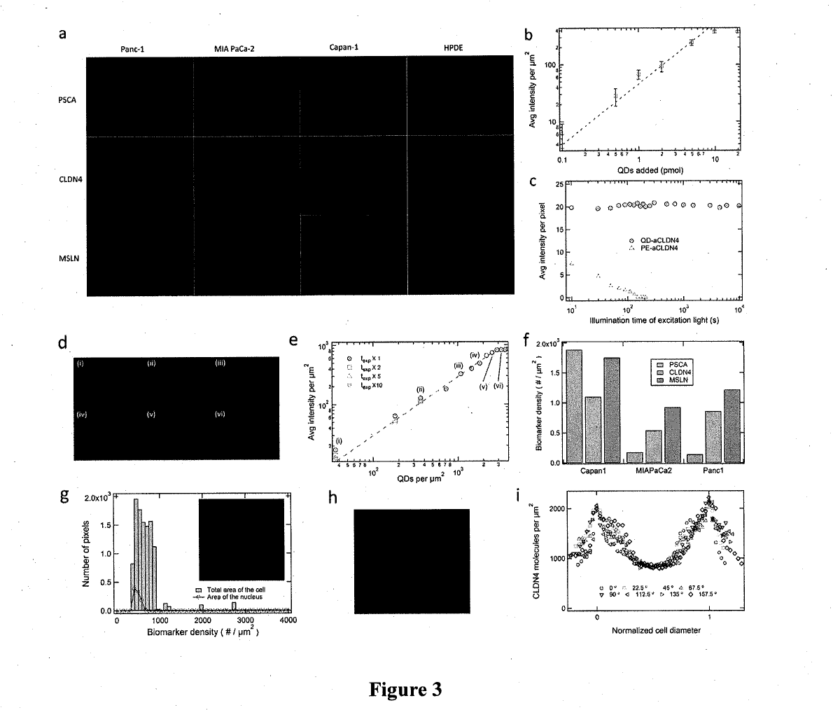

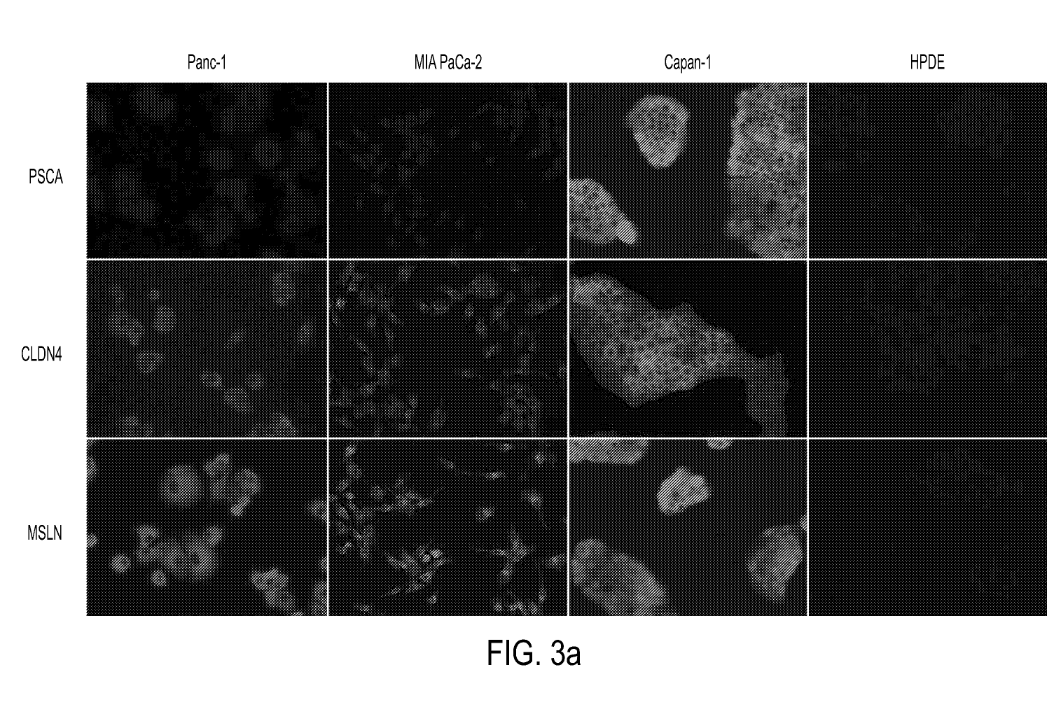

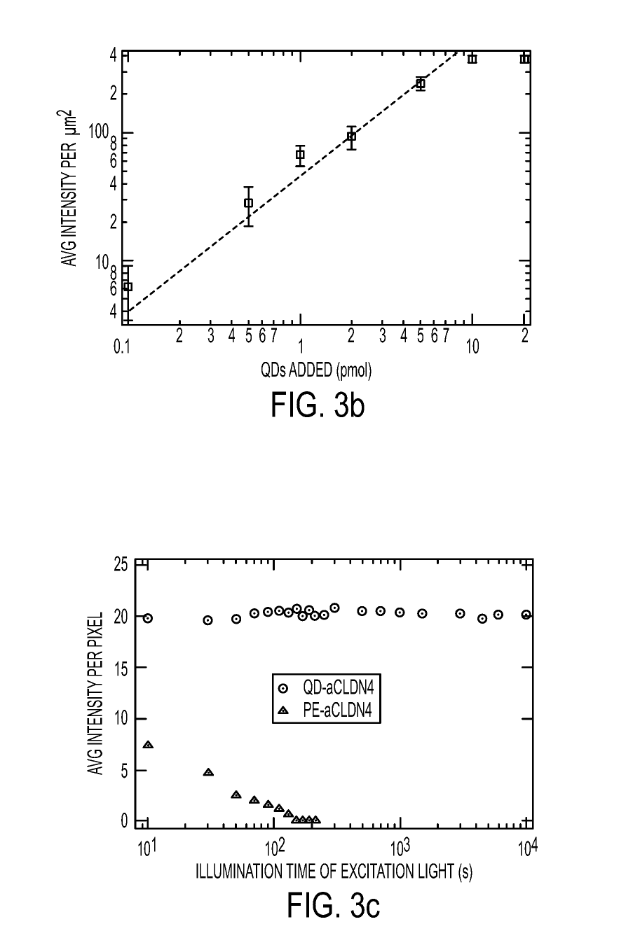

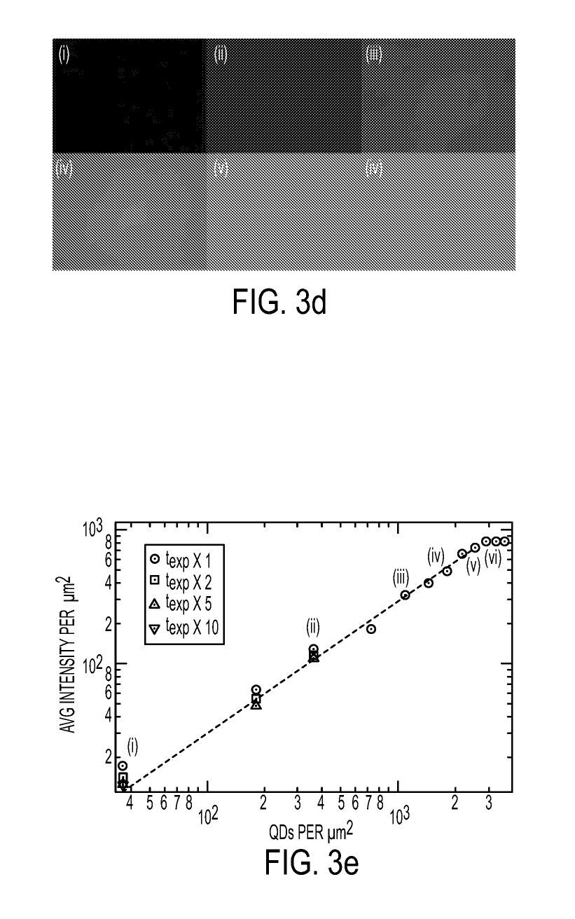

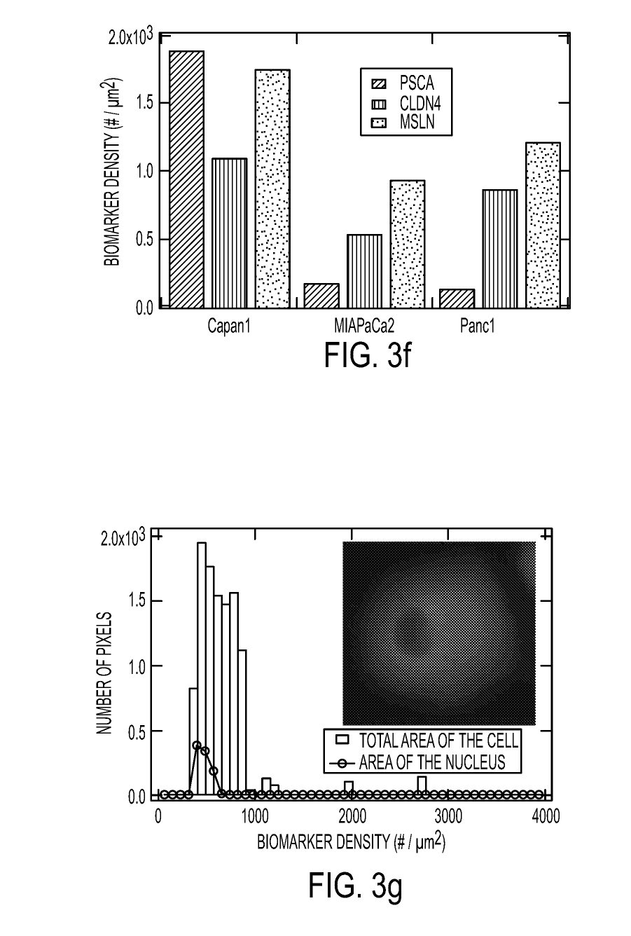

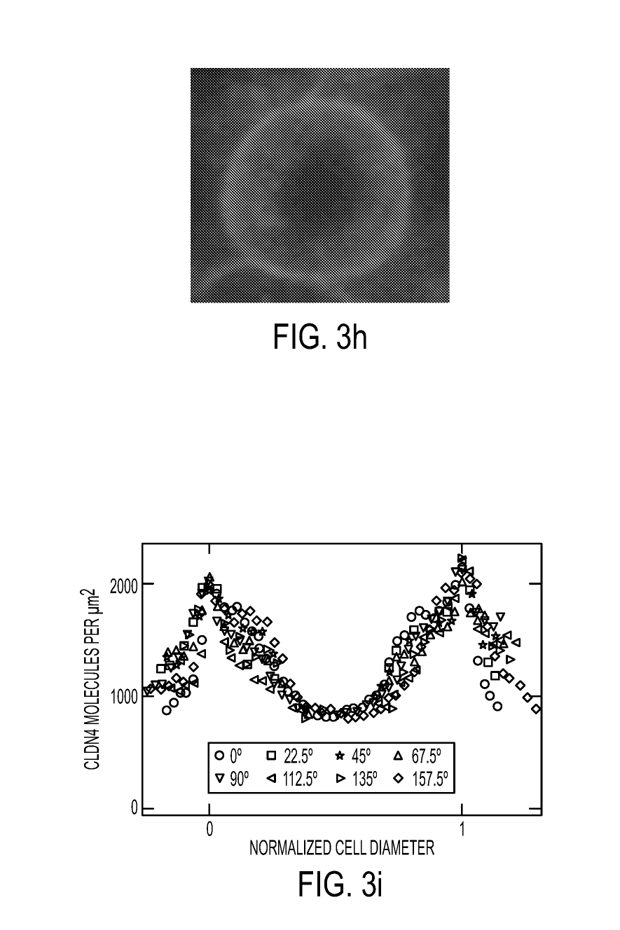

FIG. 3a shows fluorescence images for three pancreatic cancer cells lines (Panc-1, MIA PaCa-2, and Capan-1), along with a normal pancreatic duct cell line (HPDE), incubated with QD-Ab conjugates where Ab represents the antibodies to prostate stem cell antigen (PSCA), claudin-4 (CLDN4), and mesothelin (MSLN). The QDs were synthesized in our laboratories and lipid-coated prior to transferring to water. By incorporating 5 mol % amine-terminated pegylated lipids, antibodies were covalently linked to the QDs through an amide bond to lysine groups on the antibodies.

Qualitative comparison of the fluorescence images in FIG. 3a shows different levels of brightness, implying different expression levels. For example, while PSCA shows high expression in Capan-1, MSLN was strongly expressed in all three pancreatic cancer cell lines. Similarly, CLDN4 is very highly expressed in Capan-1, moderately expressed in Panc-1, and weekly in expressed MIA PaCA-2. These semi-quantitative observations are in good agreement with results from PCR, Northern blot, and Western blot reported in the literature (Michl et al., Gastroenterology 121(3):678-684, 2001; Li et al., Molecular Cancer Therapeutics 7(2):286-296, 2008; Wente et al., Pancreas 31(2):119-125, 2005). In control experiments (not shown), no fluorescence was seen for cells incubated with QDs without antibodies. We note that these results are only achieved with careful synthesis of the QD-Ab

Having established that we have saturated all biomarkers on the cells and that the fluorescence intensity is proportional to the QD concentration, we can quantitatively analyze the fluorescence images. FIG. 3f shows the average biomarker density for PSCA, claudin-4 and mesothelin in the three pancreatic cancer cell lines used in these experiments. The expression levels of these markers are in the range from 100 μm⁻² to 1900 μm⁻². Based on the relatively few quantitative studies of protein expression levels in live cells (Engelhard et al., Proc Natl Acad Sci USA, 75(11):5688-5691, 1978; Harding et al., Nature 346(6284):574-76, 1990; Kenworthy et al., J Cell Biol 142(1):69-84, 1998; Wiley et al., J Cell Biol 143(5):1317-28, 1998), this range is reasonable. For example, fluorescence resonance energy transfer microscopy showed that the surface density of glycosylphosphatidylinositol (GPI)-anchored proteins is around 10,000 μm⁻² on the apical surface of MDCK cells (Kenworthy et al., J Cell Biol 142(1):69-84, 1998). Both MSLN and PSCA belong to the family of GPI-anchored proteins.

An advantage of biomarker profiling with QD-Ab conjugates, compared to conventional methods, is that we can obtain quantitative spatial information. FIG. 3g shows the distribution of mesothelin over a single Panc-1 cell. The distribution is relatively narrow, 600±200 μm⁻² (standard deviation), indicating relatively uniform expression, as inferred from the fluorescence image. These results demonstrate that QD aggregation can be overcome with careful synthesis and design of the QD-Ab conjugates. In contrast, the distribution of claudin-4 on capan-1 cells is highly non-uniform, as known by immunofluorescence microscopy (Michl et al., Gastroenterology 121(3):678-684, 2001). These cells tend to form clusters and the intensity is much brighter at the paracellular junctions (FIG. 3h). Claudin-4 is one of the claudin family of proteins important in tight junction formation. FIG. 3i shows quantitative linear profiling of the claudin-4 density along a set of eight radial lines through the center of the cell and separated by an angle of 22.5°. In the paracellular regions, the claudin-4 density is around 2,000 μm², more than double the value in the central region. These results highlight a limitation of conventional methods such as gel-based techniques that are ensemble averages conjugates. Without appropriate functionalization and surface modification, the targeting is extremely heterogeneous on the surface of the cell and control experiments with QDs with no antibody show non-specific binding.

To quantitatively determine the expression levels we must (1) confirm that we have saturated all targeted biomarkers on the cell surface, and (2) relate the fluorescence intensity to the QDs concentration. To confirm that we have saturated all biomarkers on the cell surface, we incubated Panc-1 cells with different concentrations of QD-aMSLN conjugates and measured the average fluorescence intensity (FIG. 3b). The fluorescence intensity increases linearly with QD concentration up to about 400 μm⁻², at which point the density remains constant, indicating that all biomarkers are saturated. The fluorescence intensity is the output of the camera for the filter combination, magnification, and exposure time used in these experiments. From the slope (47.8 pmol⁻¹) and QD-Ab concentration used in our experiments (20 pmol), we can conclude that for any QD-Ab/cell line combination, all biomarkers are saturated as long as the fluorescence intensity is ≦960 μm⁻², this condition is satisfied for all biomarkers and cell lines shown in FIG. 3.

Having established that we have saturated the biomarkers, we next relate the fluorescence to the QD concentration. To quantitatively determine biomarker concentrations over a wide range requires that we vary the exposure time when capturing the fluorescence images. To do this we must consider the time dependence of the emission. FIG. 3c shows results for experiments where Panc-1 cells were incubated with QD-aCLDN4 conjugates or claudin-4 antibody conjugated with the fluorophore phycoerythrin (PE, emission 605 nm). The emission from the QD-Ab conjugates is constant for at least 10,000 s (2.8 hours) while the emission from the aCLDN-PE conjugate decreases exponentially with time due to photobleaching. This shows that we can use different exposure times for collecting the fluorescence images using QD-Ab conjugates. FIG. 3d shows fluorescence images for different concentrations of QDs between two glass slides. FIG. 3e shows that the average fluorescence intensity per μm² is linearly dependent on the QD concentration and the slope of 1.0 confirms that there are no errors in our procedure. Note that a concentration of about 40 μm⁻² is easily achieved just by increasing the exposure time from 1 s to 10 s.

### Example 3

Development of a microfluidic-based device for efficient and selective capture of pancreatic cancer cells: Enumeration of CTCs in peripheral blood of cancer patients has been reported to serve as an indicator of overall survival, disease stage forecasting, and as a promising method for clinical management (Braun et al., N Engl J Med 351(8):824-6, 2004). Nevertheless, detection of CTCs remains challenging due to their extremely low abundance among a high number of circulating blood cells. To this end, most assays employ enrichment steps based on morphometric or immunoseparation methods, which typically provide low recoveries with high purity, or low purity with high recoveries or in other cases, require complex sample processing whose success and reproducibility depend on trained personnel. Microchip technology has recently drawn much attention because of its potential to efficiently and selectively isolate and enumerate CTCs. For instance, a microfluidic approach utilizing an array of microposts coated with an anti-EpCAM antibody has been developed to capture CTCs (Nagrath et al., Nature 450(7173):1235-39, 2007). While preliminary results have been relatively successful, the major disadvantages of this device are: (1) sample processing requires several hours, (2) identification of CTCs is solely based on EpCAM, a protein not expressed by all CTCs, and (3) the recovery is about 65% and the purity is about 50%. Although another recently-developed microfluidic technique reduces the sample processing time to about 37 minutes, and claims a 97% recovery (Adams et al., J Am Chem Soc 130(27):8633-41, 2008), it is noteworthy that these data were obtained using human MCF-7 breast cancer cells, which express about 510,000 EpCAM molecules per cell, suspended in “rabbit blood”. It is well established that EpCAM is not expressed by all CTCs, and if present it is typically expressed at <50,000 molecules per cell (well within the dynamic range of our QD-Ab conjugates for biomarker profiling). Interestingly, EpCAM expression may be downregulated in the course of epithelial-mesenchymal transition (Pantel et al., Nat Rev Clin Oncol 6(4):190-191, 2009). To circumvent the aforementioned bottlenecks, we develop a high-throughput multi-channel microfluidic device presenting distinct cancer-related biomarkers for capture of CTCs with high recovery and high purity. The multi-channel platform reduces sample processing time, whereas the incorporation of distinct biological functionalities such as EpCAM, MUC1, MUC3, MUC4, MUC16 and carcinoembryonic antigen (CEA), to ensure capture of CTCs with high recovery and high purity. Enumeration of CTCs is achieved by microscopy, whereas profiling of cancer biomarkers is achieved at the single cell level using quantum dots conjugated with specific antibodies.

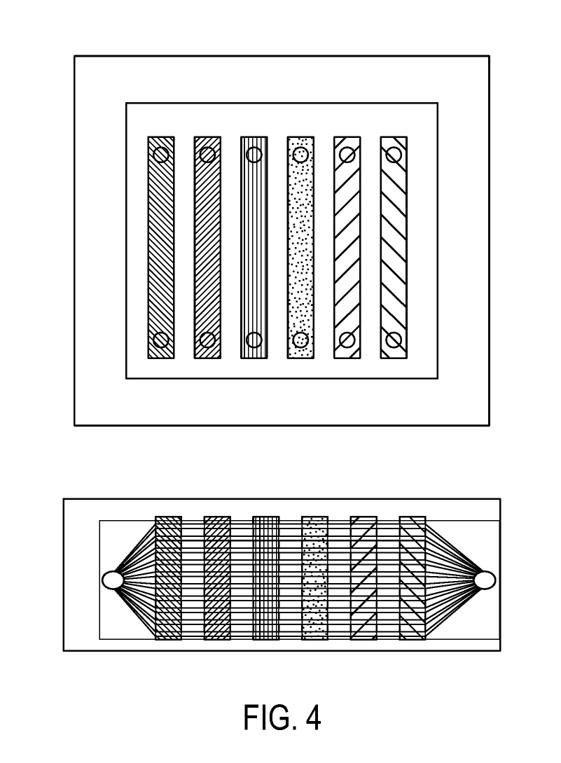

The fabrication of the microfluidic platform for CTC capture involves two steps (see FIG. 4). First, we fabricate a Microfluidic Device for Antibody Immobilization (MDAI) that will allow us to pattern patches of monoclonal antibodies (mAbs) on a glass slide. Next we fabricate a Cell Isolation Multi-channeled Microfluidic (CIMM) platform that will be located on the mAb functionalized glass slide. The channels are aligned perpendicular to the patches, such that the patch width in the MDAI is the patch length in the CIMM.

Generation of a Microfluidic Device for Antibody Immobilization (MDAI). Our objective is to immobilize a panel of mAbs specific for distinct molecular markers that are selectively or preferentially expressed by metastatic cancer cells in an effort to achieve high capture efficiency (i.e. recovery) of the heterogeneous CTCs present in peripheral blood. Since our research focuses on pancreatic cancer, we have selected the following markers that are overexpressed by pancreatic cancer cells:

Ep-CAM (epithelial cell adhesion molecule, CD326) is overexpressed in most epithelial cancers, including pancreatic cancer, but has not been demonstrated to have prognostic utility (Fong et al., J Clin Pathol 61(1):31-35, 2008).

CEA (carcinoembryonic antigen) belongs to the immunoglobulin gene superfamily of receptors, and is overexpressed in many metastatic cancer cells including pancreatic cancer cell (Allum et al., J Clin Path 39(6):610-614, 1986; Hammarstrom et al., Seminars in Cancer Biology 9(2):67-81, 1999). Interestingly, CEA expression is more prevalent in high-grade than in low-grade PanIN lesions, and has been detected in 92% of pancreatic adenocarcinoma specimens (Duxbury et al., Ann Surg 241(3):491-496, 2005).

MUC1, MUC3, MUC4, and MUC16 are members of the mucin family of high molecular weight glycoproteins (Kufe, Nature Reviews Cancer 9(12)874-885, 2009). The mucins selected for this project are all membrane proteins. MUC1 overexpression is observed during the early stages of development of pancreatic cancer, and is further increased in invasive carcinoma (Moniaux et al., Br J Cancer 91(9):1633-1638, 2004; Hruban et al., Am J Surg Pathol 28(8):977-987, 2004). MUC3 has been detected in 68%, and MUC4 has been detected in 79%, of infiltrating pancreatic adenocarcinoma (Park et al., Pancreas 26(3):c48-54, 2003). MUC16 is overexpressed in several cancers, including pancreatic cancer cells (unpublished observations), and is presently used as a marker for clinical management of ovarian cancer (Boivin et al., Gynecologic Oncology 115(3):407-413, 2009). Interestingly, a combined panel of MUC4 and MUC16 has detected 100% of late-stage ovarian cancer cases (Chauhan et al., Modern Pathology 19(10):1386-1394, 2006).

We fabricate a multichannel (L×W×H: 2 cm×200-5000 mm×25 μm) Microfluidic Device for Antibody Immobilization (MDAI) by standard photolithography. To this end, a transparency printout with multiple parallel straight channels is created, and used as a mask for fabricating a photoresist mold on a silicon wafer. PDMS pre-polymer is cast over the mold to generate the negative replica of the mold and hence the microfluidic platform (Ghosh et al., Langmuir 24(15):8134-42, 2008).

A microscope slide is pre-treated with octadecyltrichlorosilane (OTS) to render the slide surface hydrophobic so as to ensure maximum physisorption of mAbs (Ghosh et al., Langmuir 24(15):8134-42, 2008). The MDAI is reversibly assembled and aligned perpendicularly onto the OTS-treated glass slide, as shown in FIG. 4. Select antibodies are then be introduced by capillary action into the six distinct channels by dispensing 10-100 μL of an antibody at each inlet source. Subsequently, the MDAI is incubated for an hour at 37° C. under humid conditions in a CO₂ cell culture incubator to ensure maximal physisorption. Unbound antibodies are then washed off by infusing D-PBS buffer through the microchannels via the use of a microsyringe. After careful disassembly of the MDAI from the glass slide, the latter is incubated with a 2-5% polyethylene glycol (PEG) solution to passivate the remaining regions on the glass slide and eliminate non-specific binding of blood cells. To confirm the immobilization of the different antibodies on the micropatterned surface, fluorophore (FITC)-conjugated secondary antibodies are used to detect the presence of the primary mAbs by comparing the fluorescence signal on the active microregions relative to that of the PEG-functionalized inert regions.

Fabrication of a Cell Isolation Multi-channeled Microfluidic (CIMM) Platform. The fundamental purpose of utilizing a microfluidic device is to process small volumes of fluid, typically in the range of nanoliter to microliter. However, in the current application, processing of milliliters of blood specimens from cancer patients is required in view of observations showing the presence of 1-10 CTCs per mL of blood. To accomplish this high-throughput capacity, previous studies have used complicated and cumbersome microfabrication techniques, such as hot embossing (Adams et al., J Am Chem Soc 130(27):8633-41, 2008) or deep reactive ion etching (Nagrath et al., Nature 450(7173):1235-39, 2007). Instead, we employ standard photolithography to fabricate a cell isolation multi-channeled microfluidic (CIMM) platform (FIG. 4 (bottom)) capable of processing milliliters of blood within a short (about 30 minutes) time frame. To fabricate the CIMM platform, a transparency printout of the design output from modeling studies will be produced, and used as a mask for the fabrication of the PDMS-based microfluidic device, as described for the fabrication of the MDAI. The CIMM platform is assembled on top of the antibody patterned glass slide. Cell suspensions are placed at the inlet of the CIMM, whereas PVDF tubing will connect the outlet of the CIMM with a syringe pump, which will be used to perfuse cells over the micropatterned surface at prescribed flow rates. The channels are then flushed with D-PBS solution before imaged by microscopy.

### Example 4

### Optimization of the MDAI-CIMM Platforms for Efficient and High-throughput Capture of Pancreatic Cancer Cells.

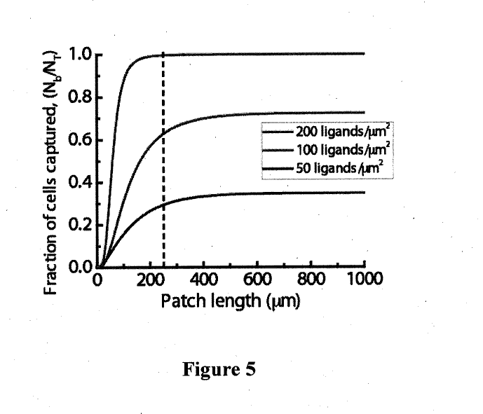

Experiments are performed using a panel of readily available metastatic pancreatic cancer cell lines, such as SW1990 and Mia PaCa-2 cells, suspended in a 3-4% dextran solution (Liang et al., Ann Biomed Eng 36(4):661-71, 2008) or 8-10% Ficoll solution (Jadhav et al., Am J Cell Physiol 283(4):C1133-43, 2002) to mimic the viscosity of whole blood. Pancreatic cell capture to micropatterned surfaces coated with high concentrations of antibodies against EpCAM, MUC1, MUC3, MUC4, MUC16 and CEA (FIG. 4) are recorded as a function of wall shear stress. The 2-D association rate (A_{c}mᵣkₒₙ) and number of bonds, n, mediating cell capture isdetermined using our mathematical model. The unstressed off rate (k^{o}_{off}) and reactive compliance (X_{β}) of individual antibody-ligand pairs (e.g. anti-EpCAM antibody binding to immunopurified EpCAM) is determined by single-molecule force spectroscopy, as has been extensively described (Hanley et al., J Biol Chem 278(12):10556-61, 2003; Hanley et al., J Cell Sci 177:2503-11, 2004; Dobrowsky et al., Methods Cell Biol 89:411-32, 2008; Panorchan et al., J Cell Sci 119:66-74, 2006). Having determined the aforementioned kinetic and micromechanical properties of the individual antibody-ligand pairs, the critical patch length required to mediate at least 99.5% of target CTCs is determined by our model, as shown in FIG. 5 for the P-selectin-PSGL-1 binding interaction. In brief, the input parameters in our model are now: (1) the association rate (A_{c}mᵣkₒₙ) and the number of bonds (n), (2) the force (F_{b}) exerted on the bond of the cell, which will be estimated using the Goldman model (Alon et al., Nature 374(6522):539-42, 1995; Goldman et al., Chem Eng Sci 22(4):653, 1967), (3) the length of the lever arm, which can be approximated by the lengths of individual adhesion molecules, (4) the receptor (mᵣ) and ligand (mₗ) site densities, and (5) k^{o}_{off} and x_{β}.

Validation of the MDAI-CIMM platforms for efficient and high-throughput capture of pancreatic cancer cells. The aforementioned experimental and analytical approach enable us to determine the optimal length of the different antibody coated-patches on the MDAI platform. As a next step, we mix prescribed numbers (1-100 cells/mL) of pancreatic cancer cell lines, pre-stained with the green Cell Tracker 5-chloromethylfluorescein diacetate (CMFDA) (Jadhav et al., J Immunol 167(10):5986-93, 2001), with citrate-anticoagulated peripheral blood isolated from human healthy volunteers. These specimens are perfused through the CIMM platform. The depth/height of the CIMM platform (25 μm) ensures that a single cell file is perfused over the antibody-coated microdomains. Detection and enumeration of captured pancreatic cancer cells is performed by fluorescence microscopy. Knowing the number of pancreatic cancer cells in blood specimens, we can readily determine the recovery and purity of our system as well as its detection limit. Purity is assessed by dividing the number of fluorescently labeled captured pancreatic cancer cells by the total number of adherent cells, which is determined by phase-contrast microscopy. A more sophisticated method of determining purity involves the use of QDs conjugated with an anti-CD45 mAb, which detects leukocytes.

### Example 5

### Quantitative Profiling of Biomarkers for Pancreatic Cancer

Tumor cells are known to express biomarkers that are characteristic of the stage of progression. We quantitatively profile a broad range of biomarkers for pancreatic cancer. These profiles provide the means to confirm the identity of pancreatic cancer derived CTCs in our combined trapping and profiling assay, and reduce the potential for false positives. In earlier studies we have demonstrated quantitative profiling of mesothelin, claudin-4, and PSCA in pancreatic cancer cells.

Molecular Markers. The following biomarkers are used to profile the pancreatic cancer cell lines: Prostate Stem Cell Antigen (PSCA), Mucin-1 (MUC1), Mucin-4 (MUC4), Mucin-16 (MUC4), claudin-4 (CLDN4), mesothelin (MSLN), CEA (epithelial cell adhesion molecule), and epithelial cell adhesion molecule (Ep-CAM).

PSCA is a gylcosylphosphatidyl inositol (GPI)-anchored protein overexpressed in adenocarcinomas Maitra et al., Mod Pathol 15(1):137A, 2002; Maitra et al. Mod Pathol 16(9):902-912, 2003; Argani et al., Cancer Res 61(11):4320-4324, 2001; Wente et al., Pancreas 31(2):119-125, 2005), and has been observed in 60% of invasive adenocarcinomas (Argani et al., Cancer Res 61(11):4320-4324, 2001).

Claudin-4 is one of a large family of tight junction membrane proteins that is overexpressed in ovarian, breast, prostate, and pancreatic tumors (Michl et al., Gastroenterology 121(3):678-684, 2001; Li et al., Molecular Cancer Therapeutics 7(2):286-296, 2008); Hewitt et al., Bmc Cancer 6(186):1-8, 2006), and has been detected in 99% of primary pancreatic adenocarcinomas (Nichols et al., Am J Clin Pathol 121(2):226-230, 2004).

Mesothelin is a GPI-anchored membrane protein overexpressed in ovarian and pancreatic cancers, and in mesotheliomas (Maitra et al., Mod Pathol 15(1):137A, 2002; Argani et al., Cancer Res 61(11):4320-4324, 2001; Hassan et al., Clin Cancer Res 8(11):3520-6, 2002). Mesothelin expression is a late event in the progression model of pancreas cancer (Maitra et al., Mod Pathol 16(9):901-912, 2003) and has been observed in 100% of primary pancreatic adenocarcinomas (Argani et al., Clin Can Res 7(12):3862-3868, 2001).

CD45 (leukocyte common antigen) is a pan-leukocyte marker commonly used to for enrichment of blood samples for CTC detection. CD45 can be used to identify any false positives (Mostert et al., Cancer Treatment Reviews 35(5):463-474, 2009).

Cell Lines. A panel of four human pancreatic cancer cell lines (Mia PaCa-2, Panc-1, Capan-1, and SW1990) are utilized for profiling studies with functionalized QDs. These are all well established pancreatic ductal adenocarcinoma lines and serve as models of advanced disease (Tan et al., Cancer Invest 4(1):15-23, 1986; Yunis et al., Int J Cancer 19(1):128-35, 1977; Fong et al., J Clin Pathol 61(1):31-5, 2008; Boivin et al., Gynecologic Oncology 115(3)407-413, 2009; Lieber et al., Int J Cancer 15(5):741-7, 1975; Fogh et al., J Natl Cancer 58(2):209-214, 1977). The immortalized pancreatic cell line HPDE (human pancreatic duct epithelium) is used as a control (Liu et al., Am J Pathol 153(1):263-269, 1998; Furukawa et al., Am J Pathol 148(6):1763-1770, 1996).

QD-Ab Conjugates. CdSe QDs with a ZnS shell are synthesized as described (Park et al., J Phys Chem 112(46):17849-17854, 2008; Galloway et al., Science of Advanced Materials 1(1):1-8, 2009)). For most experiments we use CdS/ZnS QDs with a diameter of 7.2 nm QDs, an emission peak at about 610 nm, a FWHM of about 30 nm, and a QY in excess of 40%. To obtain high stability and monodispersity in water, the QDs are functionalized with a lipid layer (Cormode et al., Nano Lett 8(11):3715-3723, 2008; Carion et al., Nat Protoc 2(10)2383-2390, 2007; Dubertret et al., Science 298(5599):1759-1762, 2002). The hydrophobic capping ligands on the QDs after synthesis (HDA and TOP) drive the formation of a lipid monolayer, analogous to the outer leaflet in a bilayer membrane. For control experiments, QDs are coated with MHPC and DSPE-PEG2k (no antibodies). With the lipid coating, the QD diameter increases to about 13 nm in diameter, as expected for the addition of a 2.5 nm lipid. By using zwitterionic lipids, the QDs are almost electrically neutral, with a zeta potential of less than 2 mV. The targeting antibodies were conjugated to the lipid-coated QDs by incorporating an amine-terminated pegylated lipid (DSPE-PEG2k-amine). Introduction of 5 mol % of the amine-peg-lipid does not influence the hydrodynamic diameter, but does result in a small positive surface charge, as seen from the small increase in zeta potential to about 6 mV. The antibodies were covalently conjugated to the QDs through formation of an amide bond between the amine of the pegylated lipids and carboxylic acids on the antibodies. Based on the antibody to QD ratio in bulk solution, we expect an average of 3 antibodies per QD. In separate experiments (not shown), we separated the antibody fragments not covalently linked to the QDs and determined that that 66% of the antibodies conjugated to the QDs were active.

Antibody conjugation resulted in an increase in the hydrodynamic diameter of the QDs from about 13 nm to about 21 nm (for a-PSCA) and a small decrease in zeta potential. The sharp size distribution and absence of aggregates is characteristic of successful conjugation and is crucial for quantitative profiling. The absorbance/emission spectra and the quantum yield of the QDs were not influenced by conjugation and the quantum yield remained more than 40%. With careful removal of excess reagents and subsequent filtration, the QDs are stable in water for at least a few months showing no change in optical properties.

Multiplexing. We profile single cells with QD-Ab conjugates, exploiting the fact that we can synthesize QDs with different emission wavelength by tuning their size. In preliminary studies (not shown) we have demonstrated successful multiplexing with three markers: QD(Em.517 nm)-aCLDN4, QD(Em.610 nm)-aMSLN, and QD(Em.706 nm)-aPSCA. Our CdSe/ZnS QDs have a narrow emission peak, about 30 nm full width at half-maximum (FWHM), and an accessible range of emission wavelengths from 450-650 nm (Park et al., J Physical Chem 112(46):17849-17854, 2008; Galloway et al., Science of Advanced Materials 1(1):1-8, 2009). In addition, we are synthesizing CuInSe/ZnS QDs with emission wavelength from 600-850 nm although the FWHM is much larger (about 100 nm). CuInSe/ZnS QD-Ab conjugates were used in the multiplexing experiment described above (Em.706 nm) confirming that our antibody conjugation is easily transferred to a different QD system.

Imaging. Briefly, about 10⁵ cells (see above for description of cell lines) are pre-seeded in a 12-well culture dish. At 50-70% confluence (1-2 days), the cell medium is aspirated and the cells washed with HPSS and PBS. 1 mL of 3.7% formaldehyde fixation solution in PBS is added to each well for 20 min and the cells washed three times with PBS. 1 mL of 10% horse serum blocking solution in PBS is introduced to each well for 30 min and then aspirated. 0.5 mL of QD-Ab solution (typically 20 pmol of QD-Ab) is added to each well and the cells incubated at a fixed temperature for a fixed time. Next, the QD-Ab solution is aspirated and the cells washed with PBS three times. Fresh PBS or mounting solution (90% glycerol in PBS) is added to the wells prior to phase contrast and fluorescence imaging (Nikon ECLIPSE TE2000-U, excitation: 350/50 or 484/15, emission: 620/60).

Image Analysis. Immunofluorescence images are acquired and analyzed using NIS-Elements AR 3.0 software. We determine quantitatively (1) the fraction of cells targeted by counting the number of cells exhibiting fluorescence above a threshold value, (2) the uniformity of targeting by analyzing the fluorescence intensity in each pixel across individual cells, and (3) the uniformity of targeting from cell to cell by measuring the total intensity from individual cells.

Validation. The quantitative biomarker expression levels is compared to the ensemble average values obtained by flow cytometry using standard methods. A fluorohore-labeled antibody is incubated with the panel of cells used in this study and the fluorescence intensity measured in the cytometer. The average biomarker concentration is determined by attaching the fluorophore (phycoerythrin, PE) to polymer beads at different concentrations and the average intensity for each concentration used as a calibration curve.

### Example 6

Quantitative profiling of biomarkers in pancreatic cancer cells in a microfluidic platform. Fabrication of microfluidic channels. Arrays of channels are created using the approach described previously. The glass slide that serves as the base for the microfluidic channels is coated with fibronectin. Cells are plated in the channels until they have spread on the fibronectin-coated surface. Next, QD-Ab conjugates are perfused into the channels. Quantitative analysis of selected biomarkers are determined using the approach described above.

We have already solved the key technological challenges associated with the synthesis of water soluble QD-Ab conjugates and our results demonstrate quantitative profiling for a limited panel of biomarkers.

### Example 7

Enumeration and Quantitative Profiling of Pancreatic Cancer Cells and Ctcs from Clinical Samples.

The major advantages of our technology are: (1) high efficiency recovery, (2) high accuracy by reducing or even eliminating any false-positive events by using, for instance, QD-CD45 mAb conjugates to identify leukocytes, and (3) quantitative profiling of cancer-related biomarkers at the single cell level provide for the first time invaluable insights to the disease staging, forecasting, and clinical management. Peripheral blood from healthy volunteers is “spiked” with prescribed numbers (1-100 cells/mL) of pancreatic cancer cell lines and perfused through the microfluidic device. Enumeration of pancreatic cancer cells is performed as described above. QD-Ab conjugates are next perfused through the device to quantitatively profile selected biomarkers on captured cells as described above.

Microfluidic platform for CTC capture. Prescribed numbers (1-100 per mL) of pancreatic cancer cells, are added to anticoagulated peripheral blood isolated from healthy human volunteers. These specimens are next perfused through the microfluidic device, and detection and enumeration of the captured pancreatic cancer cells is performed. The length of each of the 6 patches coated with a distinct mAb is selected based on our analysis above.

Quantitative profiling. After washing and enumeration of captured pancreatic cancer cells using microscopy techniques, optimal concentrations of QD-Ab conjugates are perfused through the distinct micro-channels at prescribed flow rates. QDs with different emission wavelengths are conjugated with distinct antibodies for simultaneous multiplex quantitative profiling of cancer-related biomarkers on captured cells.

Peripheral blood from pancreatic cancer patients is processed for enumeration and quantitative profiling of CTCs. For this purpose, we recruited patients under informed consent as part of a proposed Phase II trial of gemcitabine in combination with nab-paclitaxel and the Hedgehog small molecule inhibitor GDC-0449 in patients with previously untreated metastatic pancreatic cancer (NCT01088815). Blood is collected at the time of enrolment in the trial, and on two occasions following the first and second cycles of therapy.

Patient blood samples are perfused into the microfluidic platform for enumeration and biomarker profiling. CTC quantification is correlated with the baseline disease status using standard RECIST criteria, as well as objective measures of disease response (as assessed by CA19-9 levels, FDG-PET scans and CT) with each cycle of therapy. CTC numbers at baseline and following the first cycle of therapy are correlated with the progression-free and overall survival of patients by regression analysis. This well annotated clinical study forms the basis for understanding how CTC dynamics might predict the response to therapy in advanced pancreatic cancer, and allow us to segue into future trials centered on patients with resectable pancreatic cancer (i.e. in the adjuvant setting).

### Example 8

We have selected three biomarkers for pancreatic cancer for quantitative imaging: prostate stem cell antigen (PSCA), claudin-4 (CLDN4), and mesothelin (MSLN). PSCA and MSLN are gylcosylphosphatidyl inositol (GPI)-anchored proteins whereas CLDN4 is one of a large family of tight junction proteins. PSCA is overexpressed in adenocarcinomas and present in the majority of PanIN lesions beginning with early PanIN-1 (Maitra et al., Mod Pathology 15(1):137(A), 2002; Wente et al., Pancreas 31(2):119-125, 2005). Claudin-4 overexpression is observed in intermediate PanIN-2 lesions (Michl et al., Gastroenterology 121(3):678-684, 2001; Nichols et al., Am J Clin Pathology 121(2):226-230, 2004; Morin, Cancer Res 65(21):9603-9606, 2005). Mesothelin overexpression is a late event in the progression model of pancreatic cancer, almost always associated with invasion (Maitra et al., Mod Pathology 15(1):137(A), 2002; Li et al., Molecular Cancer Therapeutics 7(2):286-296, 2008; Argani et al., Clinical Cancer Research 7(12):3862-3868, 2001). All three of these biomarkers are therapeutic targets for pancreatic cancer. Quantitative profiling of these biomarkers was studied in three pancreatic cancer cell lines: Panc-1 (derived from pancreatic ductal adenocarcinoma), MIA PaCa-2 (derived from epithelial pancreatic carcinoma cells), and Capan-1 (derived from a liver metastasis of a grade II pancreatic adenocarcinoma). The immortalized pancreatic ductal cell line HPDE was used for comparison.

QDs exhibit size-dependent absorption and emission properties (Brus, J Chemical Physics 80(9):4403-4409, 1984), high fluorescence quantum yields, and with careful functionalization have been widely used for imaging and sensing (Michalet et al., Science 307(5709):538-544, 2005; Medintz et al., Nature Materials 4(6):435-446, 2005; Gao et al., Nature Biotechnology 22(8):969-976, 2004; Sapsford et al., Sensors 6(8):925-953, 2006; Choi et al., Nat Biotechnol 25(10):1165-1170, 2007; Gao et al., Bioconjug Chem 21(4):604-609, 2010; Fu et al., Current Opinion in Neurobiology 15(5):568-575, 2005; Ballou et al., Bioconjugate Chemistry 15(1):79-86, 2004; Smith, et al., Nano Letters 8(9):2599-2606, 2008). Quantitative QD-Ab targeting requires that each target molecule (e.g. membrane protein) is conjugated with one QD and that non-specific binding is minimized. Although various functionalization schemes have been reported in the literature (Medintz et al., Nature Materials 4(6):435-446, 2005; Gao et al., Nature Biotechnology 22(8):969-976, 2004; Dubertret et al., Science 298(5599):1759-1762, 2002; Liu et al., Acs Nano 4(5):2755-2765, 2010; Howarth et al., Nature Methods 5(5):397-399, 2008; Mulder et al. Accounts of Chemical Research 42(7):904-914, 2009; Louie et al., Chemical Reviews 110(5):3146-3195, 2010), here we have developed a method based on encapsulation with a lipid layer (Dubertret et al., Science 298(5599):1759-1762, 2002; Cormode et al., Nano Letters 8(11):3715-3723, 2008; Carion et al., Nature Protocols 2(10):2383-2390, 2007; Koole et al., Bioconjugate Chemistry 19(12):2471-2479, 2008) optimized for quantitative targeting (FIG. 6a). Since quantitative biomarker analysis using QD-conjugates has not previously been reported, we cannot compare our functionalization scheme to other methods, however, through a systematic study of functionalization parameters, we show that: (1) functionalization can be achieved with commercially available reagents, (2) the yield of the functionalization process is high, (3) the QD-conjugates are monodisperse and exhibit good stability in water, and (4) the functionalization method minimizes non-specific binding to cells.

### Methods

### Synthesis of QDs

Most experiments were performed using CdSe/(Cd,Zn)S core/shell QDs with an emission wavelength of about 610 nm (Park et al., J Physical Chem 112(46):17894-17854, 2008; Galloway et al., Science of Advanced Materials 1(1):1-8, 2009). For multiplexing experiments we synthesized CdSe/(Cd,Zn)S core/shell QDs with an emission wavelength of 524 nm and CuInSe/ZnS core/shell QDs with an emission wavelength of 707 nm.

### Water Solubilization of QDs

Water soluble QDs were obtained by forming a lipid monolayer composed of MHPC/DPPE-PEG2k (80:20 mol %) or MHPC/DPPE-PEG2k/DPPE-PEG2k-COOH (80:15:5 mole %). Typically 0.25 nmol of QDs, 4 μmol of MHPC, 0.75 μmol of DPPE-PEG2k, and 0.25 μmol of DPPE-PEG2k-COOH were dissolved in 0.3 mL of chloroform. This solution was added to 2 ml of deionized water and heated and maintained at 110° C. for 1 h under vigorous stirring to evaporate chloroform. The resulting solution was sonicated for 1 h, centrifuged, and the supernatant then passed through a syringe filter with a 200 nm PTFE membrane (VWR) to remove any aggregates or unsuspended QDs. Quantum yield measurements were performed on suspensions with about 100 pmol QDs in 4 mL DI water using a Hamamatsu C9920-02 fluorometer.

### Cell Lines

A panel of three human pancreatic cancer cell lines (MIAPaCa-2, Panc-1, and Capan-1) were utilized for these studies. Mia PaCa-2 and Panc-1 were cultured with a growth medium containing DMEM (Dulbecco's Modified Eagle's Medium) as the base medium, FBS (fetal bovine serum, 10%), and P/S (penicillin/streptomycin, 1%), and Capan-1 was cultured in IMDM (Iscove's Modified Dulbecco's Medium) supplemented with 20% FBS and 1% P/S. All three cell lines were incubated at 37° C. and in 5% CO₂. The immortalized normal pancreatic cell line HPDE (human pancreatic duct epithelium) was used as a control. HPDE cells were cultured in keratinocyte serum-free (KSF) medium supplemented by bovine pituitary extract and epidermal growth factor (Gibco-BRL, Grand Island, N.Y.).

### Antibodies and Antibody Conjugation

QDs were conjugated with one of three antibodies: anti-Prostate Stem Cell Antigen (aPSCA), anti-claudin-4 (aCLDN4), or anti-mesothelin (aMSLN). The reaction of the primary amines on the antibody with lipid-modified QDs (carboxylic acid-terminated QDs) is catalyzed by 1-ethyl-3-[3-dimethylaminopropyl]carbodiimide hydrochloride (EDC) resulting in the formation of an amide bond. In a typical reaction, 1 μM QDs was mixed with 2 mM EDC and 5 mM sulfo-NHS in 0.1 M MES (pH 6.0) and incubated for 15 minutes at room temperature with gentle mixing. The remaining unreacted EDC was quenched with the addition of 20 μL of 2-mercaptoethanol (1 M) for 10 minutes. Unreacted reagents and byproducts were removed by centrifugation in 100 kDa MWCO microcentrifuge tubes at 1000 g for 5 minutes. The activated QDs were then resuspended in 1×PBS. The activated QD stock solution was mixed with antibody solution (0.5-1 mg mL⁻¹ in PBS) to obtain a 3-6 fold molar excess of the antibodies to QDs. The reaction solution was incubated at room temperature for 2 h with gentle mixing. For control experiments QDs were prepared by coating with 80 mol % MHPC and 20 mol % PEGylated lipid DPE-PEG2k (no Ab). To remove excess reagents microfiltration was performed. To ensure that any aggregates are removed, an additional filtration step was carried out using syringe type filters (pore size: 100 nm). The QD suspensions were then characterized using UV-Vis absorption, photoluminescence (PL), dynamic light scattering (DLS), and surface charge (zeta potential).

### Imaging

Briefly, about 10⁵ cells (see above for description of cell lines) were pre-seeded in a 12-well culture dish. At 50-70% confluency (1-2 days), the cell medium was aspirated and the cells washed three times with PBS. Fixing solution (3.7% Formaldehyde) was added to the wells for 20 min and washed three times with PBS. The cells were then incubated with a blocking buffer (10% horse serum or 5% BSA in PBS) for 1 h prior to introducing 500 μL of QD-Ab conjugates to each well and then incubated at RT for 30 min. In most cases cells were incubated with 20 pmol QDs, however, in control experiments we used 0.1-20 pmol. Next, the QD-Ab solution was aspirated and the cells washed with PBS three times. The number of QDs introduced to each well (typically 20 pmol) corresponds to about 10⁸ QDs per cell. The maximum biomarker density (around 500 μm⁻²) corresponds to about 10⁵ per cell or a maximum QD excess of about 1000 QDs per biomarker.

Phase contrast and fluorescence images were taken with a Nikon ECLIPSE TE2000-U microscope equipped with a filter wheel allowing us to mix-and-match excitation and emission filters depending on the QDs (Ex: 350/50, 484/15, 555/25; Em: 457/30, 517/40 (FITC), 605/40 (TRITC), 620/40, or 665/LP). For experiments with QDs (Em. 607 nm), we used Ex: 555/25 and Em: 605/40. See Supplementary Information for QD emission and filter ranges. All images were obtained with a ×20 objective using Nikon Elements software. Images were recorded using a CoolSNAP HQ² camera with 2×2 binning yielding 696×520 pixels, and an output intensity range from 0-255. The exposure time was 0.5 s unless otherwise indicated.

### Flow Cytometry Analysis

Cell were centrifuged at 500×g for 5 mins and washed three times in an isotonic PBS buffer supplemented with 0.5% BSA to remove contaminating serum components that may be presented in the culture medium. Cells were resuspended in the same buffer to a final concentration of 4×10⁶ cells mL⁻¹ and 25 μL of cells (10⁵ cells) transferred to a test tube. 10 μL of PE-conjugated anti-human claudin-4 antibodies (IgG_{2A}) was then added to the test tube and incubated for 30 min. As a control for analysis, cells in a separate tube were treated with a PE-labeled mouse IgG_{2A} isotype control.

### Image Analysis

Immunofluorescence images were acquired and analyzed using Nikon NIS-Elements AR 3.1 software. The software was used to automatically select the cell boundaries and to generate the pixel statistics of the cellular region. The average fluorescence intensity per μm² within the cellular region was determined quantitatively, which allows us to make quantitative comparisons between different cell lines and different antibodies (i.e. different molecular biomarkers). Control experiments included: (1) PEGylated neutral-charge (zwitterionic) QD-L-PEG (no antibody) incubated with pancreatic cancer cell lines and a normal pancreas epithelial cell line (HPDE), and (2) QD-Ab conjugates incubated with HPDE cells.

### Results

### Lipid Encapsulation

The hydrophobic capping ligands on the QDs after synthesis drive the formation of a lipid monolayer, analogous to the outer leaflet in a bilayer membrane. Due to the high curvature of the QDs, a combination of single and double acyl chain phospholipids was used to form the outer leaflet. To determine the optimum composition, QDs were incubated in solution containing different concentrations of single alkyl chain phospholipid (MHPC) and double alkyl chain phosphoethanolamine lipid (DPPE). The yield of the functionalization process was higher than 60% for compositions in the range from 20 to 50 mol % DPPE. For ≦20 mol % DPPE, the QD-L conjugates are monodisperse with an average hydrodynamic diameter of about 13 nm, as expected for the addition of a 2 nm lipid to the 8 nm diameter CdSe/(Cd,Zn)S QDs. In contrast, for ≧30 mol % DPPE, the QDs were polydisperse implying that larger micelles containing multiple QDs are formed at higher concentrations of the double acyl chain phospholipid. The stability in water is also dependent on the lipid composition: QDs with 80 mol % MHPC and 20 mol % DPPE are stable for at least 100 h, significantly longer than other compositions. Replacing the DPPE with a pegylated version (DPPE-PEG2k), resulted in QD-L-PEG conjugates that were stable for several weeks. Finally, the quantum yield of QD-L conjugates was greater than 40% for QDs with 80 mol % MHPC/20 mol % DPPE, and was and significantly higher than other compositions.

### Charge and Antibody-Conjugation

Targeting antibodies were covalently conjugated to the lipid-coated QDs by incorporating a COOH-terminated pegylated lipid (DPPE-PEG2k-COOH). The introduction of charged groups increases stability: QDs that are near-neutral tend to aggregate, resulting in a very low yield after filtration. Conversely, QDs with significant charge exhibit high levels of non-specific binding to cells in control experiments. Consequently, there is an optimal range of charge (corresponding to a zeta potential of about −10 mV) to minimize aggregation, maximize yield and stability in water, and minimize non-specific binding. Using zwitterionic lipids, the QDs are almost electrically neutral, with a zeta potential of less than 2 mV (FIG. 6c). Introduction of 5 mol % of the COOH-PEG-lipid does not influence the hydrodynamic diameter (FIG. 6b) but results in a small negative surface charge, corresponding to a zeta potential of about −7 mV (FIG. 6c). The antibodies were covalently conjugated to the QDs through formation of an amide bond between the carboxylic acid of the pegylated lipids and primary amines (lysine or N-terminus) on the antibodies. In control experiments, we separated the antibody fragments not covalently linked to the QDs and determined that at least one antibody per QD was active.

Antibody conjugation resulted in an increase in the average hydrodynamic diameter of the QDs from 13 nm to about 21 nm (FIG. 6b) (for a-PSCA) and a small increase in the magnitude of the zeta potential due to the contribution from the antibodies (FIG. 6c). The sharp size distribution and absence of aggregates (FIG. 6b) is characteristic of successful conjugation and is crucial to minimizing non-specific binding for quantitative profiling. The low concentration of carboxylated PEG-lipids minimizes aggregation during antibody-conjugation and charge-induced non-specific binding. The absorbance/emission spectra (FIG. 6d) and the quantum yield (FIG. 6e) of the QDs were not influenced by conjugation and the quantum yield remained more than 40%. With careful removal of excess reagents and filtration, the QDs are stable in water for at least several weeks showing no change in optical properties.

### Profiling

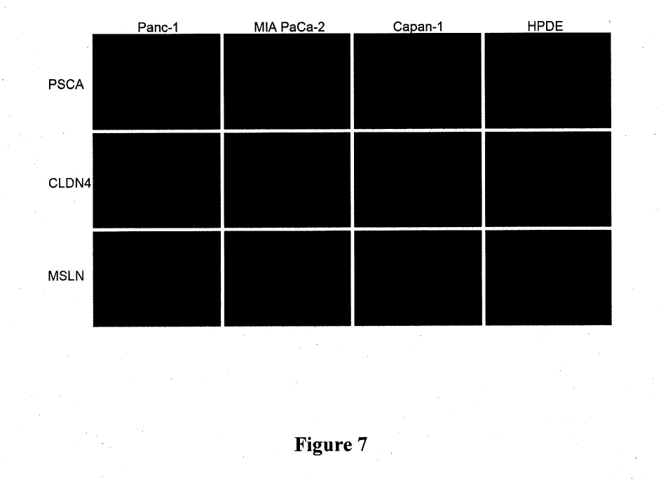

FIG. 7 shows a panel of fluorescence images after incubating Panc-1, MIA PaCa-2, and Capan-1 cells with QD-Ab conjugates. The corresponding phase contrast images are shown in Supplementary Figs. S3-S6. The absence or very low level of fluorescence for HPDE cells or cells incubated with QDs without antibodies indicates that the QD-Ab conjugates exhibit very low non-specific binding. We therefore hypothesize that the fluorescence from the pancreatic cancer cell lines is due to the binding of one QD-Ab conjugate to one target biomarker on the cell surface. This hypothesis is verified in subsequent experiments.

The fluorescence images from the Panc-1 and MIA PaCa-2 cells are very uniform, in part due to the fact that the cells are relatively isolated. In contrast, the fluorescence from the Capan-1 cells is more pronounced at the cell-cell boundaries. The spatial distribution is discussed in more detail below. Qualitative comparison of the fluorescence images in FIG. 7 shows different intensity levels, implying different expression levels. For example, while PSCA shows high expression in Capan-1, MSLN was highly expressed in all three pancreatic cancer cell lines. Similarly, CLDN4 is very highly expressed in Capan-1, moderately expressed in Panc-1, and weekly in expressed MIA PaCA-2. These semi-quantitative observations are in good agreement with results from PCR, Northern blot, and Western blot reported in the literature (Wente et al., Pancreas 31(2):119-125, 2005; Michl et al., Gastroenterology 121(3):678-684, 2001; Li et al., Molecular Cancer Therapeutics 7(2):286-296, 2008; Argani et al., Clinical Cancer Research 7(12):3862-3868, 2001). We note that these results are only achieved with careful synthesis of the QD-Ab conjugates. Without appropriate functionalization and surface modification, targeting is extremely heterogeneous on the cell surface and control experiments with QDs with no antibody show significant non-specific binding.

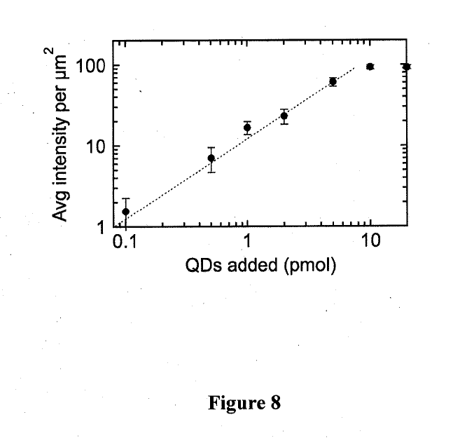

To quantitatively determine the expression levels we must (1) confirm that we have saturated all targeted biomarkers on the cell surface and (2) relate the fluorescence intensity to the QD concentration. To confirm that we have saturated all biomarkers on the cell surface, we incubated Panc-1 cells with different concentrations of QD-L-aMSLN conjugates and measured the average fluorescence intensity per cell (FIG. 8). The fluorescence intensity increases linearly with QD concentration up to 10 pmol, at which point the fluorescence intensity remains constant, indicating that all biomarkers are saturated. Prior to saturation, the slope is 1.0 confirming negligible non-specific binding and no competition for binding sites. Finally, we can conclude that for any QD-Ab/cell line combination, all biomarkers are saturated as long as the fluorescence intensity is ≦240 μm⁻², and this condition is satisfied for all biomarkers and cell lines shown in FIG. 7.

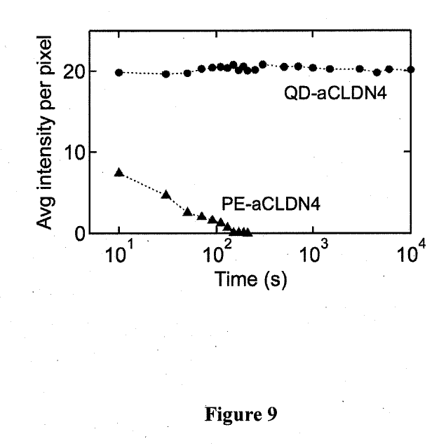

Having established that we have saturated the biomarkers on the cell surface, we next relate the fluorescence intensity to the QD concentration. To quantitatively determine biomarker concentrations over a wide range requires that we vary the exposure time when capturing the fluorescence images. To do this we must consider the time dependence of the emission. FIG. 9 shows results for experiments where Panc-1 cells were incubated with QD-L-aCLDN4 conjugates or claudin-4 antibody conjugated with the fluorophore phycoerythrin (PE, emission 605 nm), PE-aCLDN4. The emission from QD-L-aCLDN4 is constant for at least 10⁴ s while the emission from the PE-aCLDN4 conjugates decreases exponentially with time due to photobleaching. The stable emission for the QDs shows that we can linearly scale fluorescence intensities from different exposure times. Photobleaching results in an exponential decrease in emission for the PE-aCLDN4 conjugates and highlights the difficulty in using fluorophores for quantitative analysis (Resch-Genger et al., Nature Methods 5(9):763-775, 2008).

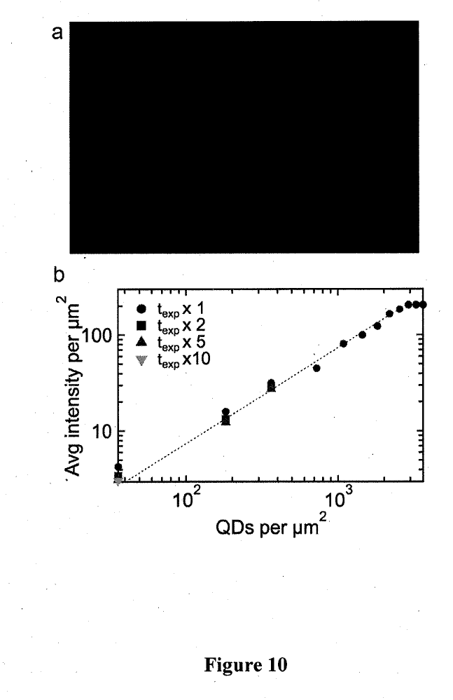

To relate the fluorescence intensity to QD concentration, a fixed volume of QD suspension was located between two glass slides (FIG. 10a). By confining the area of the suspension between the glass slides we can relate the fluorescence intensity to an areal density of QDs (FIG. 10b). The average fluorescence intensity per unit area is linearly dependent on the QD concentration and the slope of 1.0 confirms that there are no errors in our procedure.

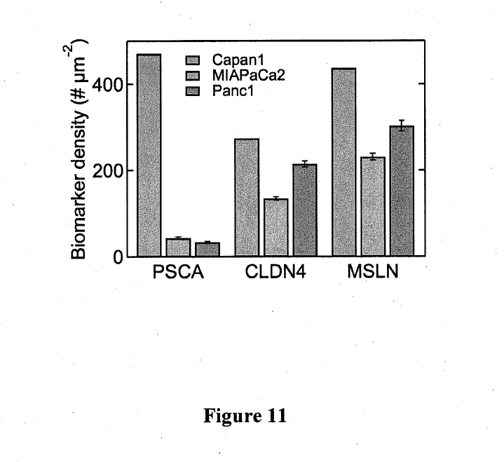

Having established that we have saturated all biomarkers on the cells and that the fluorescence intensity is proportional to the QD concentration, we can quantitatively analyze the fluorescence images. FIG. 11 shows the average biomarker density for PSCA, claudin-4 and mesothelin in the three pancreatic cancer cell lines. The expression levels of these markers are in the range from about 30 μm⁻² to 470 μm⁻². The expression levels for CLDN4 and MSLN on HPDE cells were less than 15 μm⁻² while the expression level for PSCA was about 44 μm⁻². From analysis of the background intensity we determined a detection limit of about ±4 μm⁻² (SD). The emission from cells incubated with QDs without targeting antibodies (QD-L-PEG) corresponds to an average level of non-specific binding of 15 μm⁻², just above the detection limit.

To validate the biomarker densities we performed flow cytometer analysis for CLDN4 expression on MIA PaCa-2 cells with phycoerythrin (PE)-conjugated anti-CLDN4. From control experiments with beads conjugated with known concentrations of PE and the known ratio of PE to antibodies, the number of PE molecules per cell was converted to antibodies per cell. From flow cytometry analysis we obtain an average CLDN4 density on MIA PaCa-2 cells of 121±0.15 μm⁻² (SE, N=5000 cells), in excellent agreement with the value of 135±3.6 μm⁻² obtained from QD-aCLDN4 conjugates (average expression level per cell, N=100 cells).

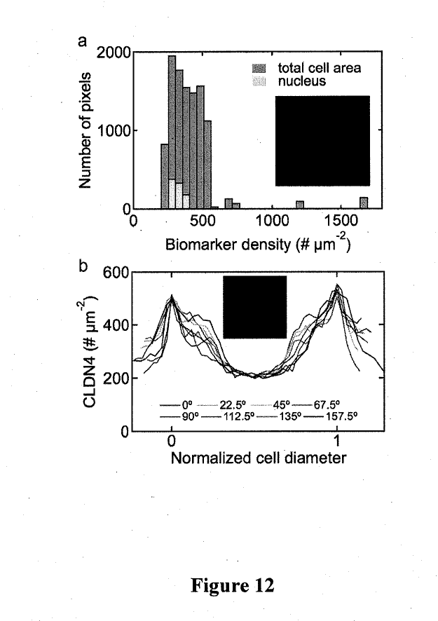

An advantage of biomarker profiling with QD-Ab conjugates, compared to conventional methods such as flow cytometry, is that we can obtain quantitative spatial information. FIG. 12a shows the distribution of mesothelin over a Panc-1 cell. The distribution over the single cell is relatively narrow, 304±0.5 μm⁻² (SE, N=10,802 pixels) indicating relatively uniform expression as inferred from the fluorescence image (inset). These results also demonstrate that QD aggregation and non-specific binding can be overcome with careful synthesis and design. In contrast, the distribution of claudin-4 on capan-1 cells is highly non-uniform, as known from previous studies using immunofluorescence microscopy (Michl et al., Gastroenterology 121(3):678-684, 2001). These cells tend to form clusters and the intensity is much brighter at the paracellular junctions. FIG. 12b shows quantitative linear profiling of the claudin-4 density along a set of eight radial lines through the center of the cell and separated by an angle of 22.5°. In the paracellular regions, the claudin-4 density is around 500 μm⁻², more than double the value in the central region.

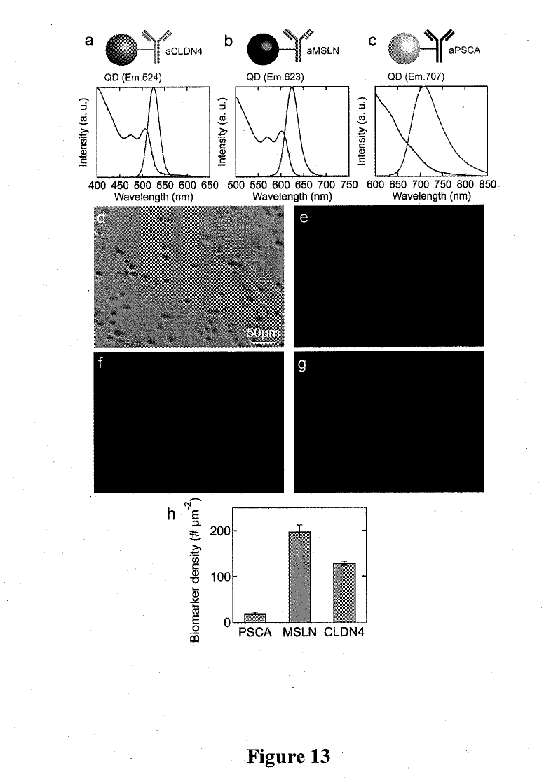

So far we have demonstrated quantitative profiling at the single cell level and spatial profiling. For high throughput profiling of multiple biomarkers, it would be desirable to perform multiplexed imaging. By attaching different antibodies to QDs with different emission wavelength, we prepared color-coded QD-Ab conjugates (see FIG. 13) to demonstrate multiplexed targeting in human pancreatic cancer cell lines: QD(Em.524 nm)-L-aCLDN4 (green), QD(Em.623 nm)-L-aMSLN (red), and QD(Em.707 nm)-L-aPSCA (NIR). FIG. 13 shows the absorbance and emission spectra for each of the color-coded QD-Ab conjugates. The wavelength of each QD was tuned to minimize the overlap of the emission with those of other QDs, but still to be detectable using different emission filters. Equal amounts of the three different color-coded QDs were simultaneously incubated with MIA PaCa-2 cells and FIG. 13 shows the resulting phase contrast image and fluorescence images at the same location taken with different emission filters. Biomarker densities determined from quantitative analysis of the fluorescence images (FIG. 13), are in a good agreement with the results from the individual QD-Ab conjugates (FIG. 7) and analysis (FIG. 13).

### Discussion

We have demonstrated quantitative profiling of biomarkers for pancreatic cancer at the single cell level using QD-Ab conjugates. Non-specific binding is negligible due to careful synthesis and passivation of the CdSe/ZnS QDs, lipid coating for solubility in water, and antibody coupling using pegylated lipids. The expression levels for PSCA, CLDN4, and MSLN in Capan-1, MIA PaCa-2, and Panc-1 cells are in the range from about 30 μm⁻² to 470 μm⁻². The results are in agreement with results from western blot, northern blot, and PCR where expression levels were scored on a relative scale. The highest expression levels were obtained from PSCA and MSLN in Capan-1 cells, and the lowest expression levels were for PSCA in MIA PaCa-2 and Panc-1 cells. Expression levels were validated using flow cytometry to determine the average expression levels for CLDN4 on MIA PaCa-2 cells. The determination of quantitative expression levels allows direct comparison between cell types at the single cell level. Furthermore, we can provide quantitative spatial information on the distribution of biomarkers.

We have also demonstrated quantitative multiplexed imaging using color-coded QDs. The expression levels obtained from multiplexed profiling of PSCA, CLDN4, and MSLN in MIA PaCa-2 cells very in excellent agreement with expression levels obtained from single QD-Ab experiments. These results show the feasibility of this technology for staging and forecasting since PSCA, CLDN4, and MSLN are expressed in different stages of progression of pancreatic cancer.

What is claimed as new and desired to be protected by Letters Patent of the United States is:

1. A microfluidic device for the capture, enumeration and profiling of circulating tumor cells comprising a microfluidic device for antibody immobilization (MDAI) and a cell isolation multi-channel microfluidic (CIMM) platform to form a microchannel comprising an inlet and an outlet wherein each microchannel of the device contains a binding partner for a circulating tumor cell (CTC) on the surface of the MDAI.

2. The microfluidic device of claim 1 wherein said binding partner is an antibody for the biomarker selected from the group consisting of EpCAM, MUC1, MUC3, MUC4, MUC16 and CEA.

3. A method for determining the presence of cancer in a subject comprising the steps of (i) introduction of a biological specimen, taken from the subject, through the inlet of the microfluidic device for the capture, enumeration and profiling of circulating tumor cells comprising a MDAI and a CIMM platform to form a microchannel comprising an inlet and an outlet wherein each microchannel of the device contains a binding partner for a circulating tumor cell (CTC) on the surface of the MDAI, (ii) capture of a CTC by a binding partner, (iii) binding of the captured CTC with a quantum dot-antibody indicative of cancer and (iv) identifying the biomarker bound by the quantum dot-antibody.

4. The method of claim 3, wherein said cancer is pancreatic cancer.

5. The method of claim 3, wherein the quantum dot-antibody is for a biomarker selected from the group consisting of PSCA, CLDN4 and MSLN.

6. The method of claim 3, wherein the quantum dot-antibody bound cell is quantified.

7. A method for diagnosing cancer in a subject comprising the steps of (i) introduction of a biological specimen, taken from the subject, through the inlet of the microfluidic device for the capture, enumeration and profiling of circulating tumor cells comprising a MDAI and a CIMM platform to form a microchannel comprising an inlet and an outlet wherein each microchannel of the device contains a binding partner for a circulating tumor cell (CTC) on the surface of the MDAI, (ii) capture of a CTC by a binding partner, (iii) binding of the captured CTC with a quantum dot-antibody indicative of cancer and (iv) identifying the biomarker bound by the quantum dot-antibody.

8. The method of claim 7, wherein said cancer is pancreatic cancer.

9. The method of claim 7, wherein the quantum dot-antibody is for a biomarker selected from the group consisting of PSCA, CLDN4 and MSLN.

10. The method of claim 7, wherein the quantum dot-antibody bound cell is quantified.

11. A method of monitoring the progress of treatment of cancer in a subject with cancer comprising the steps of (i) introduction of a biological specimen, taken from the subject, through the inlet of the microfluidic device for the capture, enumeration and profiling of circulating tumor cells comprising a MDAI and a CIMM platform to form a microchannel comprising an inlet and an outlet wherein each microchannel of the device contains a binding partner for a circulating tumor cell (CTC) on the surface of the MDAI, (ii) capture of a CTC by a binding partner, (iii) binding of the captured CTC with a quantum dot-antibody indicative of cancer and (iv) identifying the biomarker bound by the quantum dot-antibody.

12. The method of claim 11, wherein said cancer is pancreatic cancer.

13. The method of claim 11, wherein the quantum dot-antibody is for a biomarker selected from the group consisting of PSCA, CLDN4 and MSLN.

14. The method of claim 11, wherein the quantum dot-antibody bound cell is quantified.

15. A kit comprising the microfluidic device of claim 1 and a plurality of quantum dot-antibody conjugates.

16. A microfluidic device for the capture, enumeration and profiling of circulating tumor cells, comprising:
a substrate;
a region of binding material attached to said substrate;
a channel layer attached to said substrate such that said channel layer, said substrate and said region of binding material define a microfluidic channel having an inlet, an outlet and a channel region that passes over at least a portion of said region of binding material, wherein said binding material comprises a binding partner for a circulating tumor cell.

17. The microfluidic device according to claim 16, wherein said binding partner is an antibody for a biomarker corresponding to said circulating tumor cell selected from the group consisting of EpCAM, MUC1, MUC3, MUC4, MUC16 and CEA.

18. The microfluidic device according to claim 16, wherein said microfluidic channel has a cross-sectional dimension that is sufficiently large to allow single biological cells of interest to pass through and sufficiently small to exclude multiple biological cells from simultaneously passing through.

19. The microfluidic device according to claim 16, further comprising a plurality of regions of binding material attached to said substrate, wherein said microfluidic channel passes over predetermined lengths of each of said plurality of regions of binding material.

20. The microfluidic device according to claim 16, wherein said channel layer, said substrate and said plurality of regions of binding materials define a plurality of microfluidic channels, wherein each of said plurality of microfluidic channels passes over predetermined lengths of each of said plurality of regions of binding material.

21. A method for the capture, enumeration and profiling of circulating tumor cells, comprising:
passing a sample through a microfluidic channel, wherein said microfluidic channel comprises at least a section with a binding material attached thereto, said binding material comprising a binding partner for a circulating tumor cell (CTC) such that circulating tumor cells attach to said section of said microfluidic channel providing attached CTCs;
passing a solution through said microfluidic channel, subsequent to said passing said sample through said microfluidic channel, said solution comprising quantum dot-antibodies that selectively attach to said attached CTCs to provide labeled CTCs; and
illuminating said microfluidic channel with light to cause said labeled CTCs to emitted light for detection and analysis.
